(19) United States
(12) Patent Application Publication
(10) Pub. No.: US 2013/0203726 A1
(43) Pub. Date: Aug. 8, 2013

(54) FtsZ INHIBITORS AS POTENTIATORS OF BETA-LACTAM ANTIBIOTICS AGAINST METHICILLIN-RESISTANT STAPHYLOCOCCUS

(71) Applicants: Terry Roemer, Cranford, NJ (US); Sang Ho Lee, Belleville, NJ (US); Lisa Wang Jarantow, Collegeville, PA (US)

(72) Inventors: Terry Roemer, Cranford, NJ (US); Sang Ho Lee, Belleville, NJ (US); Lisa Wang Jarantow, Collegeville, PA (US)

(21) Appl. No.: 13/583,480

(22) PCT Filed: Mar. 4, 2011
(86) PCT No.: PCT/US11/27136

§ 371 Date: Nov. 19, 2012

Related U.S. Application Data
(60) Provisional application No. 61/312,068, filed on Mar. 9, 2010.

(51) Int. Cl. A61K 31/545; A61K 31/397; A61K 31/4025; A61K 31/437

(52) U.S. Cl. CPC A61K 31/545; A61K 31/437; A61K 31/397; A61K 31/4025
USPC 514/207; 514/210.09; 514/210.13; 514/209

(57) ABSTRACT

The present invention relates to the use of inhibitors of FtsZ, an ancestral tubulin of prokaryotes, to restore susceptibility to β-lactam antibiotics, including carbapenems and cephalosporins, particularly in methicillin-resistant Staphylococcus aureus (MRSA), methicillin-resistant Staphyloccus epidermis (MRSE), and other coagulase negative staphylococci (MRCNS).

### CROSS-REFERENCE TO RELATED APPLICATIONS

Not Applicable

### FIELD OF THE INVENTION

The present invention relates to the use of inhibitors of FtsZ, an ancestral tubulin of prokaryotes, to restore susceptibility to β-lactam antibiotics, including carbapenems and cephalosporins, particularly in methicillin-resistant Staphylococcus aureus (MRSA), methicillin-resistant Staphyloccus epidermidis (MRSE), and other methicillin-resistant coagulase negative staphylococci (MRCNS).

### BACKGROUND OF THE INVENTION

β-lactam antibiotics interfere with the assembly of peptidoglycan in the bacterial cell wall by inhibiting enzymatic reactions involved in the final stages of assembly. β-lactams antibiotics are among the most widely used antibiotics due to their relatively high effectiveness and low side effects. See Wilke et al., 2005, Curr Opin Microbiol 8:525-533. However, drug resistance is a major problem with β-lactam antibiotics. For example, MRSA is a major cause of nosocomial and community-acquired illnesses throughout the world and accounts for ˜60% of all staphylococcal infections. Infection with MRSA results in diverse clinical manifestations ranging from minor skin and soft tissue infections to life threatening endocarditis, bacteremia and pneumonia. Due to the prevalence of resistance mechanisms in MRSA and other bacteria, new ways of overcoming this resistance, especially through unique combinations of antibiotic targets, are desirable.

A particular protein class that was recently defined as a potential new target for antibiotics is the class of FtsZ proteins. See Desai et al., 1998, BioEssays 20:523-527. FtsZ is broadly conserved across Gram negative and Gram positive pathogens. It is an essential gene involved in cell division and encodes an ancestral tubulin specific to bacteria. See Adams et al., 2009, Nature Reviews in Microbiology 7:642-651 The FtsZ gene product is a guaninine-nucleotide binding protein thought to polymerise in a GTP-regulated manner into structures similar to those formed after tubulin polymerisation. During cell-division, FtsZ localizes to a constricting ring structure at the division site to coordinate localized cell wall synthesis and cell division.

A number of inhibitors of FtsZ have been described. See Haydon et al., 2008, Science 321:1673-1675; Margalit et al., 2004, Proc Natl Acad Sci USA 101:11821-11826; and Stokes et al., 2005, J Biol Chem 280:39709-39715.

Accordingly, there is a need for new approaches to target methicillin-resistant Staphylococcus which will restore the effectiveness of β-lactam antibiotics.

Citation or identification of any reference in this section or any other section of this application shall not be construed as an indication that such reference is available as prior art to the present invention.

### SUMMARY OF THE INVENTION

The present invention relates to the use of an inhibitor of FtsZ in combination with a β-lactam antibiotic to potentiate the in vivo effects of the β-lactam antibiotic for the treatment of bacterial infections, for example, those arising from bacteria which are resistant to one or more antibacterial agents, e.g., methicillin-resistant S. aureus, methicillin-resistant S. epidermidis and other methicillin-resistant coagulase negative staphylococci.

Accordingly, in one aspect, the invention provides a method for treating a methicillin-resistant Staphylococcal infection in a patient, preferably a human, where the treatment includes administering a therapeutically effective amount of a combination of 1) a β-lactam antibiotic; and 2) an inhibitor of FtsZ. The β-lactam antibiotic may be a carbapenem, cephalosporin, monobactam or penicillin. Exemplary carbapenem antibiotics useful in the methods of the invention include ertapenem, imipenem and meropenem. In some embodiments of the invention, the β-lactam may be administered with a β-lactamase inhibitor. In some embodiments of the invention, the carbapenem may be administered with a DHP inhibitor, e.g., cilastatin. Exemplary cephalosporins include cefepime, ceftazidime, cefriaxone, cefazolin, and cefdinir. Exemplary penicillins include piperacillin and amoxicillin.

In certain embodiments of the invention, the inhibitor of FtsZ inhibitor is selected from the group consisting of 2-carbamoyl pteridine, 534F6, A-189, amikacin, GTP, PC58538, PC170942, PC175515, PC175568, PC190723, SRI-3072, a zantrin, and analogs thereof. In other embodiments, the inhibitor of FtsZ is selected from the group consisting of berberine, cinnamaldehyde, curcumin, dichamanetin, sanguinarine, taxane, totaro, viriditoxin, xanthoradone, and analogs thereof.

Additionally, in other embodiments of the invention, the inhibitor of FtsZ is selected from the group which interfere with of ZipA-FtsZ protein interactions, including indole[2,3-a]quinolizin-7-ones (See Jennings et al., 2004, Bioorg. Med. Chem.14:1427-1431), substituted 3-(2-indolyl)piperidines and 2-phenyl indoles (See Jennings et al., 2004, Bioorg. Med. Chem.12:5115-5131) or carboxybiphenylindole inhibitors (See Sutherland et al., 2003, Org. Biomol. Chem. 1:4138-4140).

In the various embodiments of the invention, the β-lactam antibiotic and inhibitor of FtsZ can be administered sequentially or concurrently. Preferably, the β-lactam antibiotic and inhibitor of FtsZ are administered together. When administered concurrently, the β-lactam antibiotic and inhibitor of FtsZ may be administered in the same formulation or in separate formulations. When administered sequentially, either the β-lactam or inhibitor of FtsZ may be administered first. After administration of the first compound, the other compound is preferably administered within 1, 2, 3, 4, 5, 10, 15, 30, or 60 minutes. In one aspect of the invention, when a β-lactamase inhibitor is used, it may be administered separately, or in a formulation with the inhibitor of FtsZ and/or β-lactam antibiotic. In one aspect of the invention, when a DHP inhibitor is used to improve the stability of a carbapenem, it may be administered separately, or in a formulation with the inhibitor of FtsZ and/or carbapenem.

In certain embodiments, the combination of an inhibitor of FtsZ and a β-lactam antibiotic has a synergistic effect on antibacterial activity. In other embodiments, the combination of an inhibitor of FtsZ and β-lactam antibiotic is effective to restore susceptibility of the methicillin-resistant Staphylococci to the β-lactam antibiotic.

The invention further provides pharmaceutical compositions comprising of a β-lactam antibiotic, an inhibitor of FtsZ, and a pharmaceutically acceptable carrier. The β-lactam antibiotic and the inhibitor of FtsZ are present in such amounts that their combination restores sensitivity of a methicillin-resistant Staphylococcus strain to a β-lactam antibiotic. In embodiments where the β-lactam antibiotic is a carbapenem, the carbapenem antibiotic can be selected from the group consisting of ertapenem, imipenem and meropenem. In embodiments where the β-lactam antibiotic is a cephalosporin, the cephalosporin can be selected from the group consisting of cefepime, ceftazidime, cefriaxone, cefazolin, and cefdinir. In embodiments where the β-lactam antibiotic is a penicillin, the penicillin can be selected from piperacillin and amoxicillin. In certain embodiments, the composition further comprises a β-lactamase antibiotic.

In other embodiments where the β-lactam antibiotic is a carbapenem, the invention further provides pharmaceutical compositions comprising a carbapenem antibiotic, a DHP inhibitor, an inhibitor of FtsZ, and a pharmaceutically acceptable carrier.

### BRIEF DESCRIPTION OF THE DRAWINGS

FIG. 1. Depletion of FtsZ, FtsA, or FtsW restores MRSA susceptibility to β-lactam antibiotics. MRSA COL strains bearing xylose-regulated antisense fragments corresponding to FtsZ (SACOL1199; left panels), FtsA (SACOL1198; middle panels), or FtsW (SACOL2075; right panels) were seeded in LB agar with 50 mM xylose (top panels) or no xylose (bottom panels) to partially induce or repress antisense expression, respectively. Drugs were spotted at numerically indicated positions as follows: (1) imipenem; 12.8 μg, (2) ertapenem; 51.2 μg, (3) cefepime; 102.4 μg, (4) DMSO; 10 μl of a 20% stock, (5) 0.1 M PBS (pH 6) and 10 mM MOPS (pH 7) with 10 μl of each buffer spotted adjacent to one another, (6) vancomycin; 0.8 μg, (7) ceftazidime; 102.4 μg, (8) ceftriaxone; 102.4 μg, and (9) piperacillin combined with the β-lactamase, tazobactam in a 8:1 molar ratio, 25.6 μg. Note, identical spotting conditions and positioning were applied to all plates.

FIGS. 2A-D. Checkerboard analysis of PC190723 in combination with imipenem and ertapenem against MRSA COL and MRSE CLB 26329 clinical isolates. A. imipenern/MRSA COL; B. ertapenem/MRSA COL; C. imipenem/MRSE CLB 26329; D. ertapenem/MRSE CLB 26239. Drug concentrations and fractional inhibitory concentration (FIC) are indicated. Drug combinations demonstrating significant synergy (FICI≦0.5) are indicated by shading. Arrows indicate PC190723 FIC values sufficient to restore imipenem and ertapenem activity to their respective clinical breakpoint MIC.

FIGS. 3A-E. Checkerboard analysis of PC190723 and Zantrin Z3 in combination with cefepime (A), ceftazidime (B), cefazolin (C), cefdinir (D), and amoxicillin (E) against MRSA clinical isolate COL. Inhibitory concentrations are plotted as the fractional inhibitory concentration (FIC) for each agent. Synergism is achieved by the combination of the two agents fully inhibiting growth provided their individual drug concentrations sum to a FICI value ≦0.5 (indicated by the diagonal line).

FIGS. 4A-E. Checkerboard analysis of PC190723 and Zantrin Z3 in combination with cefepime (A), ceftazidime (B), cefazolin (C), cefdinir (D), and amoxicillin (E) against MRSE clinical isolate CLB 26329. Inhibitory concentrations are plotted as the fractional inhibitory contentration (FIC) for each agent. Synergism is achieved by the combination of the two agents fully inhibiting growth provided their individual drug concentrations sum to a FICI value ≦0.5 (indicated by the diagonal line).

FIG. 5. Checkerboard assay summary of PC190723 synergy in combination with β-lactams against MRSA and MRSE clinical isolates, COL and CLB26329, respectively. Synergism is achieved by the combination of the two agents fully inhibiting growth provided their individual drug concentrations sum to fractional inhibitor concentration index (FICI) of ≦0.5. Shaded boxes indicate significant synergies between PC190723 and β-lactams when PC190723 FIC=0.25. Checkerboard assays were performed according to CLSI standard protocols. Note: PC190723 MIC=1.0 vs MRSA and MRSE.

FIG. 6. Checkerboard assay summary of zantrin Z3 synergy in combination with β-lactams against MRSA and MRSE clinical isolates, COL and CLB26329, respectively. Synergism is achieved by the combination of the two agents fully inhibiting growth provided their individual drug concentrations sum to fractional inhibitor concentration index (FICI) of ≦0.5. Shaded boxes indicate significant synergies between Z3 and various β-lactams when Z3 FIC=0.25. Checkerboard assays were performed according to CLSI standard protocols. Note: Z3 MIC=8−16.0 μg/ml vs MRSA and MRSE

FIGS. 7A-B. PC190723 in vivo synergy and efficacy in a deep thigh model of MRSA COL infection. Two independent studies (FIGS. 7A, 7B) illustrate that combination therapy utilizing nonefficacious doses of imipenem and PC190723 dose-dependently synergize to reduce bacterial burden in vivo.

### DETAILED DESCRIPTION OF THE INVENTION

The present invention is based, in part, on Applicants' genetic and pharmacological validation that inhibition of the bacterial FtsZ gene product restores MRSA susceptibility to β-lactams, including carbapenems. Known FtsZ inhibitors, and inhibitors identified through, for example, target-based whole cell assays and in vitro biochemical assays, can be used as drugs to be used in combination with β-lactam antibiotics to regain efficacy against methicillin-resistant Staphylococcal species such as MRSA, MRSE and MRCNS. They can also be used as a starting point to optimize their synergistic effect with β-lactam antibiotics for the development of new drugs. Structurally unrelated inhibitors of FtsZ are useful as potentiators of β-lactam antibiotics in that they provide unexpectedly superior activity when used in combination with a β-lactam antibiotic, particularly against methicillin resistant strains and species of Staphylococcus.

An inhibitor of FtsZ is a potentiator of β-lactam antibiotics useful in the treatment of bacterial infections, e.g., infections with MRSA, in that it demonstrates synergistic antibacterial activity when used in combination with β-lactam antibiotics. Antibacterial activity can be evidenced by cell killing or inhibition of growth or any other means known in the art. In one aspect of the invention, the inhibitor of FtsZ can enhance the activity of a β-lactam antibacterial agent by inducing susceptibility (by overcoming resistance) to the antibacterial agent in a drug-resistant strain such as MRSA or MRSE. In another aspect of the invention, the inhibitor of FtsZ can enhance the activity of a β-lactam antibacterial agent by reducing the dosage of the antibacterial agent need for a therapeutic effect in a drug-sensitive strain. For example, if an inhibitor of FtsZ reduces the Minimum Inhibitory Concentration (MIC) of an antibacterial agent (where the MIC is the minimum concentration of antibacterial agent which will completely inhibit growth) in a susceptible strain, then such treatment may be advantageous to enable a reduction in the amount of antibacterial agent administered (could reduce side effects of an antibiotic), or to decrease the frequency of administration. In another aspect of the invention, inhibitors of FtsZ can complement the activity of a β-lactam antibacterial agent to prevent the emergence of a resistant sub-population in a heterogeneous bacterial population with a resistant sub-population.

Treatments using inhibitors of FtsZ as a potentiator represent a new approach to antibacterial therapy in which an inhibitor of FtsZ can be administered together with a β-lactam antibiotic (either concurrently or sequentially) to allow effective treatment of an infection involving a resistant bacterium. Inhibitors of FtsZ can be used to enhance the activity of antibacterial agents whose clinical efficacy has been limited by the increasing prevalence of resistant strains.

The compounds of the present invention are useful per se and in their pharmaceutically acceptable salt and ester forms are potentiators for the treatment of bacterial infections in animal and human subjects, particularly those involving methicillin-resistant Staphylococcal strains. As used herein, the term “pharmaceutically acceptable ester, salt or hydrate”, refers to those salts, esters and hydrated forms of the compounds disclosed herein which would be apparent to the pharmaceutical chemist. i.e., those which are substantially non-toxic and which may favorably affect the pharmacokinetic properties of said compounds, such as palatability, absorption, distribution, metabolism and excretion. Other factors, more practical in nature, which are also important in the selection, are cost of the raw materials, ease of crystallization, yield, stability, solubility, hygroscopicity and flowability of the resulting bulk drug.

Preferred examples of a salt include alkali metal salts such as a sodium salt, a potassium salt and a lithium salt; alkaline earth metal salts such as a calcium salt and a magnesium salt; metal salts such as an aluminium salt, an iron salt, a zinc salt, a copper salt, a nickel salt and a cobalt salt; amine salts such as inorganic salts such as an ammonium salt and organic salts such as a benzylamine salt, a chloroprocaine salt, a dibenzylamine salt, a dibenzylethylenediamine salt, a dicyclohexylamine salt, a diethanolamine salt, a diethylamine salt, an ethylenediamine salt, a glucosamine salt, a guanidine salt, a morpholine salt, an N-benzyl-phenethylamine salt, an N-methylglucamine salt, an N,N-dibenzylethylenediamine salt, a phenylglycine alkyl ester salt, a piperazine salt, a piperidine salt, a procaine salt, a pyrrolidine salt, a t-octylamine salt, a tetramethylammonium salt, a triethylamine salt, and a tris(hydroxymethyl)aminomethane salt; and amino acid salts such as a glycine salt, a lysine salt, an arginine salt, an ornithine salt, a glutamate or an aspartate.

Pharmaceutically acceptable salts also include acid addition salts. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydrofluoride, hydroiodide, 2-hydroxyethanesulfonate, lactate, maleate, methanesulfonate, 2-naphthalenesulfonate, nicotinate, nitrate, oxalate, p-toluenesulfonate, pamoate, pectinate, perchlorate, persulfate, 3-phenylpropionate, phosphate, picrate, pivalate, propionate, succinate, sulfate tartrate, thiocyanate, tosylate, trifluoromethanesulfonate and undecanoate.

Pharmaceutically acceptable salts can be synthesized from the compounds disclosed herein by conventional chemical methods. Generally, the salts are prepared by reacting the free acid with stoichiometric amounts or with an excess of the desired salt-forming inorganic or organic base in a suitable solvent or various combinations of solvents. Alternatively, salts can be prepared from the corresponding sodium or potassium salt of the compounds disclosed herein using conventional ion exchange processes with cation exchange resins carrying the desired salt base.

Pharmaceutically acceptable esters are such as would be readily apparent to a medicinal chemist. Included within such pharmaceutically acceptable esters are those which are hydrolyzed under physiological conditions, i.e., biolabile esters.

Biolabile esters may be suitable for oral administration, due to good absorption through the stomach or intenstinal mucosa, resistance to gastric acid degradation and other factors. Examples of biolabile esters include compounds of the form COOM in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties: acetoxymethyl, 1-acetoxyethyl, 1-acetoxypropyl, pivaloyloxymethyl, 1-isopropyloxycarbonyloxyethyl, methoxymethyl, 1-cyclohexyloxycarbonyloxyethyl, phthalidyl and (2-oxo-5-methyl-1,3-dioxolen-4-yl)methyl.

Pharmaceutically acceptable hydrate is used in the conventional sense to include the compounds of formula I in physical association with water.

As used herein, a “potentiator” or “potentiating compound” refers to a compound which has a synergistic effect on antibacterial activity when used with an antibacterial agent. Thus, a potentiator enhances the antibacterial effect of an antibacterial agent when the two compounds are used in combination. A potentiator does not have to, but may, have significant antibacterial activity when used alone at concentrations similar to its concentration in the combination use.

As used herein, “synergy” or “synergistic” refers to the effects of a combination of antibacterial agents wherein the antibacterial activity of the combination is greater than the sum of the activity of the individual antibactrial agents, particular in strains such as methicillin-resistant Staphylococcus aureus (MRSA), methicillin-resistant Staphyloccus epidermidis (MRSE), and other methicillin-resistant coagulase negative staphylococci (MRCNS). In one embodiment, synergy is defined as an FTC index of 0.5.

The invention is also intended to include all of the isomeric forms of the compounds described herein, including racemic, enantiomeric and diastereomeric forms.

Inhibitors of FtsZ

FtsZ, a bacterial analog of tubulin, is a broadly conserved protein and an essential gene that appears to be present in all prokaryotes, where it has an essential role in cell division. See Adams et al., 2009, Nature Rev in Microbial 7:642-653; Lutkenhaus, 1993, Mol. Microbiol. 9: 403-409. FtsZ polymerizes at midcell into a cytoskeletal structure designated the FtsZ ring (Z ring) that directs the process of septation and cytokinesis. See Adams et al., 2009, Nature Revs Microbial 7:642-653; Addinal et al., 1996, Mol. Microbial. 22:231-237; Bi et al., 1991, Nature 354:161-4. The formation of the Z ring through the polymerization of FtsZ supports this hypothesis. See Lutkenhaus, 1993, Mol. Microbiol. 9:403-409. One activity of this ring is to recruit other division proteins to the division site, where they participate in formation of the septum. See Lutkenhaus, 1993, Mol Microbial 9:403-409. Correct localization of S. aureus extracellular cell wall proteins, most notably penicillin binding proteins (PBPs) including Pbp2 are dependent on FtsZ function. See Pinho et al., 2003, Mol. Microbial. 50:871-881; Pinho et al., 2005, Mol. Microbiol. 55:799-807. Thus, FtsZ function has an important role in recruiting PBPs to the division site.

FtsZ has limited sequence identity to eukaryotic tubulins, suggesting that it is an ancestral homologue. See Mukherjee et al., 1994, J Bacterial 176:2754-2758. The structures of FtsZ and tubulin support this conclusion, as the structures are remarkably similar. See Lowe et al., 1998, Nature 391:203-206; Nogales et al., 1998, Nature 391:199-203. In addition to these sequence and structural similarities there is functional similarity, as both tubulin and FtsZ are GTPases that can polymerize into higher-order structures. Like tubulin's GTPase, FtsZts GTPase activity increases dramatically with the protein concentration. See Davies et al., 1990, Annu. Rev. Biochem. 59:439-473; Lu et al., 1998, Cell Motil Cytoskeleton. 40:71-86; Wang et al., 1993, Mol Microbial. 9:435-42. The FtsZ protein of Bacillus subtilis is localized at the division site and has GTPase activity that is dependent upon FtsZ concentration. See Wang et al., 1996, Mol Microbial 21:313-319. Tubulin assembles into microtubules that are composed of protofilaments, head-to-tail polymers of the ab dimer. See Desai et al., 1997, Annu. Rev. Cell Biol. 13:83-117.

Without being bound by any mechanism, any compound that inhibits FtsZ can be useful as a potentiator of a β-lactam antibiotic. Compounds that have been identified as FtsZ inhibitors include structurally diverse classes of natural products, semi-synthetic, and synthetic molecules. See Vollmer, 2006, Appl Microbiol Biotechnol 73:37-47; Kapoor and Panda, 2009, Expert Opin. Ther. Targets 13-1037-1051. Natural product inhibitors of FtsZ include, but are not limited to, berberine, cillamaldehyde, curcumin, dichametin, sanguinarine, totarol, and viriditoxin. See Kapoor and Panda, 2009, Expert Opin. Ther. Targets 13-1037-1051. Synthetic and semisynthetic inhibitors of FtsZ include, but are not limited to, 2-carbamoyl pteridine, 534F6 derivatives, A-189, Amikacin, GTP analogs, PC58538, PC170942, PC175515, PC175568, PC190723, SRI-3072, taxanes, and zantrins. See Kapoor and Panda, 2009, Expert Opin. Ther. Targets 13-1037-1051; and Stokes et al., 2005, J Biol Chem 280:39709-39715. Inhibitors of FtsZ include, but are not limited to, the compounds disclosed below, and pharmaceutically acceptable salts thereof.

Polyphenols

A number of structurally diverse polyphenolic compounds have been identified in a high throughput protein-based chemical screen to identify small molecules that target assembly-dependent GTPase activity of FtsZ including Zantrin Z1 and derivatives thereof, Zantrin Z2, Zantrin Z3, Zantrin Z4, Zantrin Z5. See Margalit et al., 2004, Proc Natl Acad Sci USA 101:11821-11826.

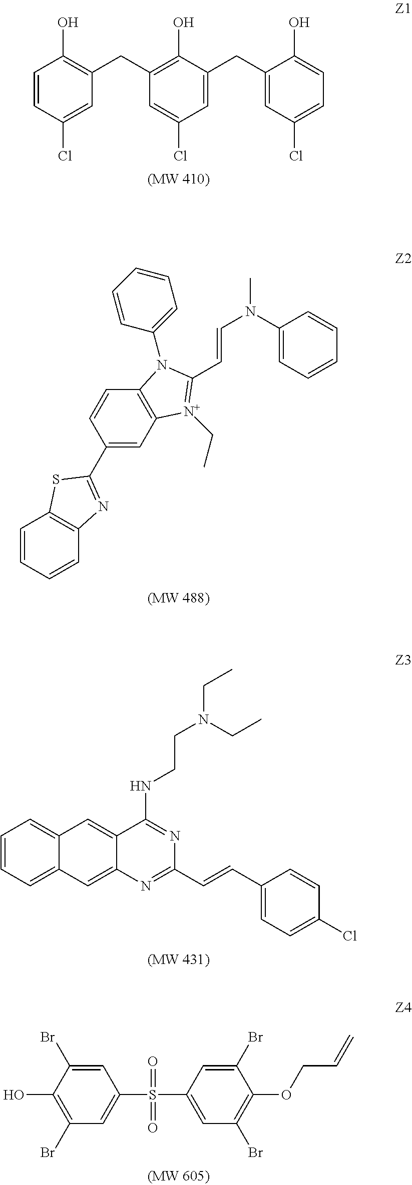 [SMILES: Oc1ccc(Cl)cc1Cc1cc(Cl)cc(Cc2cc(Cl)ccc2O)c1O ; CC[n+]1c(/C=C/N(C)c2ccccc2)n(-c2ccccc2)c2ccc(-c3nc4ccccc4s3)cc21 ; CCN(CC)CCNc1nc(/C=C/c2ccc(Cl)cc2)nc2cc3ccccc3cc12 ; C=CCOc1c(Br)cc(S(=O)(=O)c2cc(Br)c(O)c(Br)c2)cc1Br]

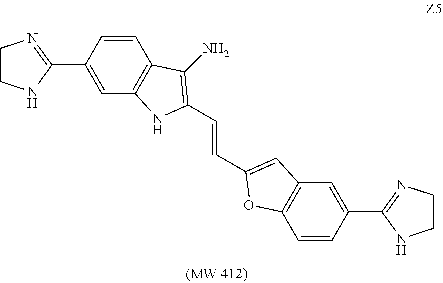 [SMILES: Nc1c(/C=C/c2cc3cc(C4=NCCN4)ccc3o2)[nH]c2cc(C3=NCCN3)ccc12]

Other polyphenolic compounds have been identified as natural products such as 2^{′″}-hydroxy-5″-benzylisovamol-B (from X. afticana) and derivatives thereof, dichamanetin (from Uvaria chamae) and derivatives thereof. See Urgaonkar et al., 2005, Organic Lett 7:5609-5612; and WO 2007/056188.

2-Alkoxycarbonylaminopyridines

A series of substituted 2-alkoxycarbonylaminopyridines were screened for inhibition of FtsZ. See international Patent Application Publication No. WO2004/005472. Several compounds were identified as FtsZ specific inhibitors of polymerization and GTP hydrolysis. Among the compounds so identified are [8-(4-diethylamino-1-methyl-butylamino)-2,3,diphenyl-pyrido[2,3-b]pyrazin-6-yl]-carbamic acid ethyl ester (SRI-3072); [5-amino-3-{[(4-methoxy-phenyl)-methyl-amino]-methyl}-1,2-dihydro-pyrido[3,4-b]pyrazin-7-yl]carbamic acid ethyl ester (SRI-5713); [5,6-diamino-4-(2-hydroxy-1-methyl-3-phenoxy-propylamino)-pyridinyl-2-yl]-carbamic acid ethyl ester (SRI-7405); (5-amino-3-butyl-2-methyl-1,2-dihydro-pyrido[3,4-b]pyrazin-7-yl)-carbamic acid ethyl ester (SRI-7462); 1-amino-8-phenyl-6,7-dihydro-5H-2,5,9-triaza-benzocyclohepten-3-yl)-carbamic acid ethyl ester (SRI-7614); [5-amino-3,(4-butylcarbamoyloxy-phenyl)-2-methyl-1,2-dihydro-pyrido[3,4-b]pyrazin-7-yl]carbamic acid ethyl ester (SRI-20158); (5-amino-2,3-diphenyl-pyrido[3,4-b]pyrazin-7-yl)-carbonic acid ethyl ester (3302-89F); (5-ethoxy-2,3-diphenyl-pyrido[3,4-b]pyrazin-7-yl)-carbamic acid ethyl ester (3491-23); [2,3-diphenyl-8-(4-sulfamoyl-benzylamino)-pyrido[2,3-b]pyrazin-6-yl]-carbamic acid ethyl ester (4427-026-15); 4-[(6-amino-2,3-diphenyl-pyrido[2,3-b]pyrazin-8-ylamino)-methyl]-N,N-diethyl-benzenesulfonamide (4427-143); and (5-amino-2,3 -dipheyl-2H-pyrido[4,3-b][1,4]oxazin-7-yl)-carbamic acid ethyl ester (CAO-040).

The structures of two representative compounds are shown below:

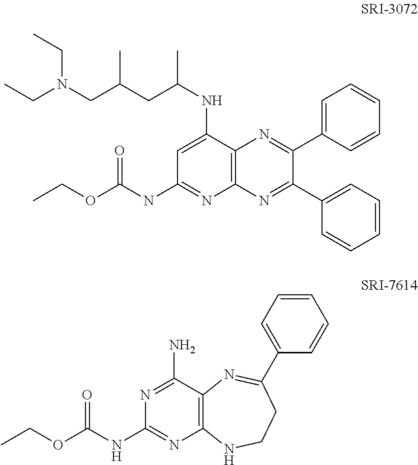 [SMILES: CCOC(=O)Nc1cc(NC(C)CC(C)CN(CC)CC)c2nc(-c3ccccc3)c(-c3ccccc3)nc2n1 ; CCOC(=O)Nc1nc(N)c2c(n1)NCCC(c1ccccc1)=N2]

Benzopheanthridine

Sanguinarine is an example of a benzophenanthridine alkaloid which was isolated from the rhizomes of Sanguinaria canadensis. See Beuria et al., 2005, Biochem 44:16584-16593. It prevents bacterial cell division by inhibiting cytokinesis. See id. Sanguinarine has been used as a lead compound for the development of FtsZ inhibitors.

Benzamides and Related Compounds

A large number of substituted benzamides, pyridylamides, were found to have antibacterial activity and thought to act by inhibiting FtsZ. See International Patent Application Publication No. WO2007/107758. This mechanism of action appears to be based in part on the activity of the substituted alkoxybenzamides, including 3-methoxybenzamide, against FtsZ. See Ohashi et al., 1999, J Bacteriol. 181:1348-51; and Czaplewski et al., 2009, Bioorg Med Chem Lett. 19:524-7.

Additional compounds including substituted benzamidines, isonicotinamidines, phenyl and pyridyl oxadiazolones, substituted thiadiazolylmethoxybenzamide or thiadiazolylmethoxypyridylamide, including PC190723, were also found to have antibacterial activity thought to be related to inhibition of FtsZ. See International Patent Application Publication Nos. WO2009/040507 and WO 2009/037485; and Hayden et al., 2008, Science 321:1673-1675. The structure of PC190723 is shown below:

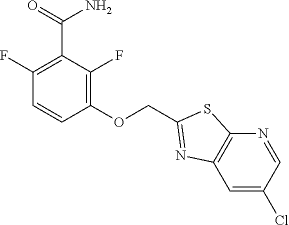 [SMILES: NC(=O)c1c(F)ccc(OCc2nc3cc(Cl)cnc3s2)c1F]

Structurally related analogs of PC190723 are disclosed in Czaplewski et al., 2009, Bioorg Med Chem Lett. 19:524-7 and Stokes et al., 2005, J Biol Chem. 280:39709-15.

Natural Products

Additional natural products have been identified which have served as leads for development for FtsZ inhibitors. These include viriditoxin (from Aspergillus sp.; see Wang et al., 2003, J Biol Chem 278:44424-8) and the structurally related class of natural product, xanthoradones (see Yamazaki et al., 2009, J. Antibiot. 62:435-7; Yamazaki et al., J Antibiot 62:431-4); berberine (a natural plant alkaloid; see Domadia et al., 2008, Biochemistry 47:3225-34); and cinnamaldehyde (a natural product derived from spices; see Domadia et al., 2007, Biochem Pharmacol 74:831-40).

Aminonaphthalenes

The hydrophobic probe, 5,5′-bis-(anilino-1-naphthalenesulfonoate (bis-ANS) has been found to inhibit FtsZ assembly. See Yu et al., 1998, J Biol Chem 273:10216-22.

FtsZ-ZipA Interaction Inhibitors

In addition to small molecules that inhibit FtsZ GTPase activity and/or polymerization, inhibitors of FtsZ association with accessory proteins required for FtsZ function have been identified, particularly against Escherichia coli FtsZ-ZipA interacting protiens. See

Kenny et al. 2003, Analytical Biochemistry 323:224-233; Tsao et al., 2006, Bioorg. Med. Chem. 14:7953-7961. These include indole [2,3-a]quinolizin-7-one inhibitors (Jennings et al., 2004, Bioorg. Med. Chem. 14:1427-1431), substituted 3-(2-indolyl)piperidines and 2-phenyl indoles (Jennings et al., 2004, Bioorg. Med. Chem.14:5115-5131), and carboxybiphenylindoles (Sutherland et al., 2003, Org. Biomol. Chem., 1:4138-4140).

Nucleic Acid Based Drugs

Nucleic acid based drugs that inhibit the expression of the FtsZ gene or translation of FtsZ mRNA can be used in the methods of the invention. Nucleic acid based drugs include, but are not limited to, antisense RNA, ribozymes, siRNA, peptide nucleic acids (see U.S. Pat. No. 6,548,651) and any others known in the art.

Nucleoside Analogs

Nucleoside analogs, particularly GTP analogs, such as 8-bromoguanosine 5′-triphosphate, can also be used in the methods of the invention. See Lappchen et al., 2005, Biochemistry 44:7879-7884, and International Patent Application Publication No. WO 01/32187.

Identification of Additional Inhibitors of FtsZ

Additional inhibitors of FtsZ can be identified using any of several assay systems known in the art for assessing inhibition of FtsZ. Caution must be used to identify inhibitors which do not target eukaryotic tubulin.

One assay is described in International Patent Application Publication No. WO2004/009835. In the assay described therein, a plasmid expressing a xylose-inducible anti-sense RNA for Staphylococcus aureus FtsZ is transfected into a S. aureus cell. The recombinant cells are grown in a nutrient medium in the presence of a test substance under conditions in which expression of the RNA fragment occurs at a level that pre-sensitizes the cell to substances that act at FtsZ. A comparison of growth is made between cells that maintain expression of the AS-RNA and those that lack the AS-RNA (revertant cells).

Another assay is described in International Patent Application Publication No. WO 03/014343. The assay described therein is a direct fluorescence detection technique based on the physical separation of fluorescently labeled polymers of FtsZ from monomeric forms. A mixture of wild-type FtsZ and fluorescently labeled mutant FtsZT65C is combined with the test compound. The polymerization is allowed to take place, after which the polymeric forms are separation form the monomeric fowls by centrifugation. The amount of fluorescence is quantified.

Various other assays are described in International Patent Application Publication No. WO 02/094976. Such assays include in vitro assays, such as an enzyme-coupled NADH fluorescence assay and a radioactive thin-layer chromatographic assay measuring conversion of radiolabeled GTP to radiolabeled GDP, and in vivo assays, such as one measuring ring assembly using a FtsZ-gfp fusion protein, one employing a temperature sensitive FtsZ mutant, and one measuring cell density. Some of the assays described therein are amenable to high throughput screening.

Another assay is described in Stokes et al., 2005, J Bial Chem 280:39709-39715. Briefly, this assay is a cell-based reporter assay utilizing a strain of Bacillus subtilis having two reporter genes using two different promoters involved at different times in sporulation. While this assay can identify compounds active in methicillin-resistant staphylococci, the assay can be readily adapted for use in methicillin-resistant staphylococci, for example, by using promoters that are involved in the FtsZ pathway. FtsZ inhibitors identified in this assay include:

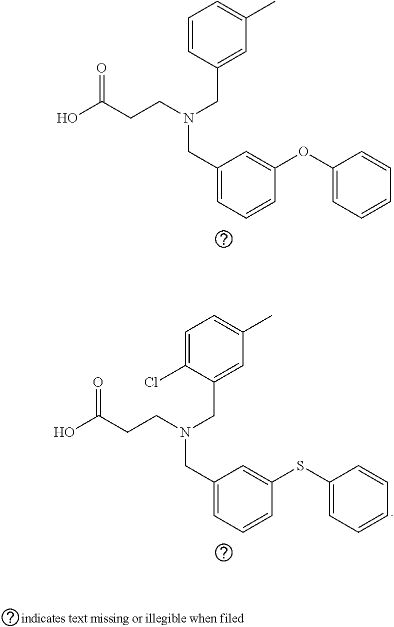 [SMILES: Cc1cccc(CN(CCC(=O)O)Cc2cccc(Oc3ccccc3)c2)c1 ; Cc1ccc(Cl)c(CN(CCC(=O)O)Cc2cccc(Sc3ccccc3)c2)c1]

β-lactam Antibiotics

β-lactams antibiotics are characterized by a 4-membered β-lactam core of consisting of three carbon atoms and one nitrogen atom. β-lactams antibiotics include carbapenems, cephalosporins, monolactams and penicillins.

Due to the activity of β-lactamases, a β-lactam antibiotic may be degradaded. Thus, in certain embodiments of the invention, a suitable β-lactamase inhibitor, such as clavunic acid, sulbactam or tazobactam, may be administered, either together or separately, with the β-lactam antibiotic. See, e.g., Drawz et al., 2010, Clin Microbiol Rev 23:160-201. The β-lactamase inhibitor should preferably be available at the desired site of action before the antibiotic to ensure immediate protection of the antibiotic.

Carbapenems

Carbapenems are a class of β-lactam antibiotics that possess the carbapenem ring system (a four-member lactam ring fused to a five member thiazolidinic secondary ring through the nitrogen and adjacent tetrahedral carbon atom). Carbapenems tend to exhibit an extremely broad spectrum of activity against gram-positive and gram-negative aerobic and anaerobic species, which is partly due to its high stability in the presence of β-lactamases. They act by binding to penicillin-binding proteins.

Carbapenems include, but are not limited to, carbapenems (including 1β-methylcarbapenems) having a side chain at the 2 position, including, but not limited to, 2-substituted alkyl-3-carboxycarbapenems (See U.S. Pat. No. 5,021,565); 2-aryl carbapenems (See U.S. Pat. No. 6,277,843); 2-(aza-9-fluorenonyl)carbapenems (See U.S. Pat. No. 5,294,610) and 2-(9-fluorenonyl)-carbapenems (See U.S. Pat. Nos. 5,034,384 and 5,025,007) including 2-(fluoren-9-on-3-yl)carbapenems containing a (bis-quaternary ammonium)methyl moiety (See U.S. Pat. No. 5,451,579); 2-benzocoumarinyl-carbapenems (See U.S. Pat. Nos. 5,216,146; 5,182,384; 5,162,314; and 5,153,186); 2-biphenyl-carbapenems (See U.S. Pat. Nos. 5,350,846; 5,192,758; 5,182,385; 5,025,006; and 5,011,832); 2-carbolinyl derivatives (See U.S. Pat. No. 5,532,261); 2-(substituted-dibenzofuranyl and dibenzothienyl) carbapenems (See U.S. Pat. Nos. 5,240,920 and 5,025,008); halophenoxy substituted carbapenems (U.S. Pat. No. 6,310,055); carbapenems with cationic —S— heteroaryl substituents (See U.S. Pat. No. 5,496,816); carbapenems having an externally alkylated mono- or bicyclic 2-quaternary heteroarylalkyl substituent (See U.S. Pat. No. 4,729,993) or carbapenems having an internally or externally alkylated mono- or bicyclic 2-quaternary heteroarylalkyl thiomethyl substituent (See U.S. Pat. No. 4,725,594); carbapenems having a 2-heteroaryliumaliphatic substituent (See U.S. Pat. No. 4,680,292); 2-naphthyl-carbapenems (See U.S. Pat. Nos. 5,006,519 and 5,032,587); 2-naphthosultam carbapenems (See U.S. Pat. Nos. 6,399,597; 6,294,529; 6,251,890; 6,140,318; 6,008,212; 5,994,345; 5,994,343; 5,756,725), e.g., 2-(naphthosultamyl)methyl-carbapenems (See U.S. Pat. No. 6,221,859), carbapenems substituted at the 2-position with a 1,1 dioxo-2,3-dihydro-naphtho[1,8-de][1,2]thiazin-2-yl group or 1,1,3 trioxo-2,3-dihydro-naphtho[1,8-de][1,2]thiazin-2-yl group linked through a CH₂ group (U.S. Pat. No. 6,346,526) or a 1,1-dioxo-2H-1-thia-2,3-diaza-naphthalene linked through a CH₂ group (See U.S. Pat. No. 6,346,525); 2-(N-imidazoliumphenyl)-carbapenems (See U.S. Pat. No. 5,276,149); 2-phenanthridinyl carbaphenems (See U.S. Pat. Nos. 5,336,674; 5,328,904; 5,214,139 and 5,153,185); 2-phenanthrenyl-carbapenems (See U.S. Pat. Nos. 5,177,202; 5,004,740 and 5,004,739); 2-phenanthridonyl-carbapenems including 2-phenanthridonyl carbapenems having cationizeable substituents (See U.S. Pat. No. 5,157,033); 2-phenyl-carbapenems (See U.S. Pat. Nos. 5,334,590 and 5,256,777), including 2-(heteroaryliumalkyl)phenyl carbapenems (See U.S. Pat. No. 4,978,659), 2-(heteroarylsubstituted)phenyl carbapenems (See U.S. Pat. No. 5,034,385), 2-(heterocyclylalkyl)phenyl carbapenems (See U.S. Pat. Nos. 5,247,074; 5,037,820 and 4,962,101), 2-(heterocyclylheteroaryliumalkyl)phenyl carbapenems (See U.S. Pat. No. 5,362,723), 2-heteroatylphenyl-carbapenems (See U.S. Pat. Nos. 5,143,914 and 5,128,335) including cationic 2-heteroarylphenyl-carbapenems (See U.S. Pat. No. 5,342,933), 2-iodo-substituted phenyl (See U.S. Pat. No. 6,255,300); (N-pyridiniumphenyl)-carbapenems (See U.S. Pat. No. 5,382,575), triazolyl and tetrazolyl phenyl substituted carbapenems (See U.S. Pat. No. 5,350,746) including 2-(1,2,3-triazolylsubstituted)phenyl carbapenems (See U.S. Pat. No. 5,208,229), and 2-(quinoliniumalkyl and isoquinoliniumalkyl)phenyl carbapenems (See U.S. Pat. Nos. 5,124,323 and 5,055,463); 2-(2-substituted pyrrolidin-4-yl) thio-carbapenems (See U.S. Pat. Nos. 5,756,765 and 5,641,770); 2-(3-pyridyl)-carbapenems (See U.S. Pat. No. 5,409,920); 2-(unsubstituted or carbon-substituted)-1-carbapen-2-em-3-carboxylic acid derivatives (See U.S. Pat. Nos. 5,258,509 and 4,775,669); carbapenems substituted at the 2-position with a 9,9-dioxo-10H -9-thia-10-aza-phenanthrene linked through a CH₂ group (See U.S. Pat. No. 6,294,528); carbapenems substituted at the 2-position with fused bi- and tricyclic 2,2-dioxo-3-X-2-thia-1-aza-cyclopenta ring systems linked through a CH₂ group (See U.S. Pat. Nos. 6,291,448 and 6,265,395); and carbapenems substituted at the 2-position with a 2-mercaptobenzothiazole moiety linked through a group —Z—CH₂— where Z represents an trans-ethenediyl group, ethynediyl group or is absent (See U.S. Pat. No. 6,288,054).

Carbapenems also include, but are not limited to, 3-phosphonate carbapenems (See U.S. Pat. No. 4,565,808); 6-amido-carbapenems (See U.S. Pat. No. 5,183,887), including, but not limited to, 6-amido-1-methyl carbapenems (See U.S. Pat. No. 5,138,050) and 6-amido-1-methyl-2-(substituted-thio)carbapenems (See U.S. Pat. No. 5,395,931); bridged carbapenems including bridged biphenyl carbapenems (See U.S. Pat. Nos. 5,401,735; 5,384,317; 5,374,630; 5,372,993); cyclic amidinyl and cyclic guanidinyl thio carbapenems (See U.S. Pat. No. 4,717,728); and tricyclic carbapenem compounds (See U.S. Pat. Nos. 6,284,753 and 6,207,823; International Patent Application Publication No. WO92/03437).

Carbapenems also include, but are not limited to, 1β-methylcarbaperiem derivatives (See U.S. Pat. Nos. 7,001,897; 6,479,478; 5,583,218; 5,208,348; 5,153,187; International Patent Application Publication Nos. WO 98/34936 and WO 99/57121; carbapenems with a carboxy subtitituted phenyl group (See U.S. Pat. No. 5,478,820); carbapenem derivatives having a substituted imidazo[5,1-b]thiazole group at the 2-position on the carbapenem ring (See U.S. Pat. Nos. 6,908,913; 6,680,313; 6,677,331; International Patent Application Publication Nos. WO 98/32760 and WO 00/06581), a substituted phenyl or a substituted thienyl directly substituted at position 3 of 7-oxo-l-azabicyclo[3.2.0]hept-2-ene (see U.S. Pat. No. 7,205,291). Carbapenems also include, but are not limited to, those disclosed in U.S. Pat. Nos. 4,943,569 and 4,888,344.

Examples of preferred carbapenems that may be used with an inhibitor of bacterial signal peptidase include, but are not limited to, imipenem, meropenem, biapenem, (4R,5S,6S)-3-[3S,5S)-5-(3-carboxyphenyl-carbamoyl)pyrrolidin-3-ylthio]-6-(1R)-1-hydroxyethyl]-4-methyl-7-oxo-1-azabicyclo[3.2.0]hept-2-ene-2-carboxylic acid, (1S,5R,6S)-2-(4-(2-(((carbamoylmethyl)-1,4-diazoniabicyclo[2.2.2]oct-1-yl)-ethyl(1,8-naphthosultam)methyl)-6-[1(R)-hydroxyethyl]-1-methyl carbapen-2-em-3-carboxylate chloride, BMS181139 ([4R-[4alpha,5beta,6beta(R*)]]-4-[2-[(aminoiminomethyl)aminolethyl]-3-[(2-cyanoethyl)thio]-6-(1-hydroxyethyl)-7-oxo-1-azabicyclo[3.2.0]hept-2-ene-2-carboxylic acid), BO2727 ([4R-3[3S*,5S*(R*)], 4alpha,5beta,6beta(R*)]]-6-(1-hydroxyethyl)-3-[[5-[1-hydroxy-3-(methylamino)propyl]-3-pyrrolidinylithio]-4-methyl-7-oxo-1-azabicyclo[3.2.0]hept-2-ene-2-carboxylic acid monohydrochloride), E1010 ((1R,5S,6S)-6-[1(R)-hydroxymethyl]-2-[2(S)-[1(R)-hydroxy-1-[pyrrolidin-3(R)-yl]methyl]pyrrolidin-4(S)-ylsulfanyl]-1-methyl-1-carba-2-penem-3-carboxylic acid hydrochloride), S4661((1R,5S,6S)-2-[(3S,5S)-5-(sulfamoylaminomethyl)pyrrolidin-3-ylithio-6-[(1R)-1-hydroxyethyl]-1-methylcarbapen-2-em-3-carboxylic acid) and (1S,5R,6S)-1-methyl-2-{7-[4-(aminocarbonylmethyl)-1,4-diazoniabicyclo(2.2.2)octan-1yl]-methyl-fluoren-9-on-3-yl]-6-(1R-hydroxyethyl)-carbapen-2-em-3-carboxylate chloride. More preferred carbapenems include, but are not limited to, biapenem, doripenem (Doribax™, Ortho-MacNeil, Raritan, N.J.), ertapenem (IVANZ®, Merck & Co., Inc., Whitehouse Station, N.J.), imipenem, meropenem (Menem®, AstraZeneca, Wilmington, Del.), panipenem, and tebipenem. In one embodiment, PRIMAXIN® (Merck & Co., Inc., Whitehouse Station, N.J.), a combination of imipenem and cilastin can be used.

Carbapenems also includes pharmaceutically acceptable salts, esters, hydrates, diastereomers and enantiomers of the compounds described above.

Carbapenems may be crystallized or recrystallized from solvents such as organic solvents forming solvates. This invention includes within its scope stoichiometric solvates including hydrates as well as compounds containing variable amounts of solvents such as water that may be produced by processes such as lyophilization. Carbapenems may be prepared in crystalline form by for example dissolution of the compound in water, preferably in the minimum quantity thereof, followed by admixing of this aqueous solution with a water miscible organic solvent such as a lower aliphatic ketone such as a di-(C₁₋₆) alkyl ketone, or a (C₁₋₆) alcohol, such as acetone or ethanol. Crystalline forms of carbepenems may also be synthesized as disclosed in U.S. Pat. No. 7,145,002.

Synthesis of carbapenems is well known in the art and is disclosed in the patents and patent applications in this section.

Cephalosporins/Cephamycins

Cephalosporins contain a nucleus of a β-lactam ring and a 6 member dihydrothiazine ring. Cephamycins contain an additional methoxy group on the β-lactam ring. Cephalosporins and cephamycines often have activity against Gram-positive or Gram-negative organisms, but are not typically active against both.

Exemplary cephalosporins include, but are not limited to, cefaclor, cefadroxil, cefadyl, cefalexin, cefamandole, cefatrizine, cefazolin, cefditoren, cefepime, cefetamet, cefdinir, cefinetazole, cefixime, cefizox, cefotaxime, cefrnetazole, cefobid, cefonicid, cefoperazone, cefotan, cefotaxime, cefotetan, cefoxitin, cefpirome, cefpodoxime, cefprozil, cefradine, cefsulodin, ceftazidime, ceftibuten, ceftidoren, ceftin, ceftizoxime, ceftriaxone, cefuroxime, cefuroxime axetil, cefzil, cephacetrile, cephalexin, 4-hydroxycephalexin, cephaloglycin, cephaloridine, cephalothin, cephamandole nafate, cephapirin, cephradine, or pharmaceutically acceptable derivatives thereof. Examples for pharmaceutically acceptable cephalosporin derivatives, which may be used are cefpodoxime proxetil, cefuroxime axetil, FK-037, 5-amino-2-[[(6R, 7R)-7-[[(Z)-2-(2-amino-4-thiazolyl)-2-methoxyimino)acetyl]amino]-2-carboxy-8-oxo-5-thia-1-azabicyclo[4.2.0]oct-2-en-3-yl]methyl]-1-(2-hydroxyethyl)-1H-pyrazolium hydroxide, inner salt, sulfate (1:1), and latamoxef (Moxalactam™).

Particularly suitable cephalosporins for co-administration with the compounds according to the invention include cefotaxime, ceftriaxone and ceftazidime, which may be used in the form of their pharmaceutically acceptable salts, for example, their sodium salts.

Penicillins

Penicillins are a class of β-lactam antibiotics that possess a β-lactam ring and a thiazolidine ring. Penicillins are used to treat susceptible, usually Gram-positive, organisms.

Exemplary penicillins include, but are not limited to, amoxicillin, amoxicillin-clavulanate, amoxycillin, ampicillin, azidocillin, azloccillin, benzathine penicillin, benzylpenicillin (penicillin g), carbenicillin, carboxypenicillin, cloxacillin, co-amoxiclav, cyclacillin, dicloxacillin, epicillin, flucloxacillin, hetacillin, mezlocillin, nafcillin, oxacillin, phenoxymethylpenicillin (penicillin v), piperacillin, pirbenicillin, pivmecillinam, procaine benzylpenicillin (procaine penicillin), propicillin, sulbenicillin, tazocin (ureidopenicillin piperacillin with the beta-lactamase inhibitor tazobactam), ticarcillin, timentin, or pharmaceutically acceptable salts thereof.

The penicillins may be used in the form of pro-drugs thereof; for example as in vivo hydrolysable esters, for example the acetoxymethyl, pivaloyloxymethyl, α-ethoxycarbonyloxy-ethyl and phthalidyl esters of ampicillin, benzylpenicillin and amoxycillin; as aldehyde or ketone adducts of penicillins containing a 6-α-aminoacetamido side chain (for example hetacillin, metampicillin and analogous derivatives of amoxycillin); and as α-esters of carbenicillin and ticarcillin, for example the phenyl and indanyl α-esters.

Particularly suitable penicillins for co-administration with the compounds according to the invention include ampicillin, amoxycillin, azlocillin, carbenicillin, mezlocillin, piperacillin, and ticarcillin. Such penicillins may be used in the form of their pharmaceutically acceptable salts, for example their sodium salts. Alternatively, ampicillin or amoxycillin may be used in the form of fine particles of the zwitterionic form (generally as ampicillin trihydrate or amoxycillin trihydrate) for use in an injectable or infusable suspension, for example, in the manner described herein in relation to the compounds of formula I. Amoxycillin, for example in the form of its sodium salt or the trihydrate, is particularly suitable for use in compositions according to the invention.

Monobactams

Monobactams have a single β-lactam core. Aztreonam is currently the only example of a monobactam.

Pharmaceutical Applications

The present invention provides methods of treating bacterial infections, particularly those involving a methicillin-resistant Staphylococcal sp., in a patient in need thereof which comprises administering, in combination with a β-lactam antibiotic, a therapeutically effective amount of an inhibitor of FtsZ (i.e., sufficient to restore susceptibility to a β-lactam antibiotic). The β-lactam antibiotic may be a carbapenem, cephalosporin/cephamycin, monolactam or penicillin. In certain embodiments, a β-lactamase inhibitor may also be administered. In embodiments where the β-lactam antibiotic is a carbapenem, in additional embodiments, the method may further comprise administering a DHP inhibitor. The inhibitor of FtsZ may be any of the compounds described above. The present invention also provides pharmaceutical compositions that can be used for therapeutic treatments. As used herein, a patient may be a mammal, e.g., a dog, cat, horse, pig, or primate. The patient may also be an adult or child. Preferably, the patient is an adult human or human child.

As used herein, “therapeutically effective” amount generally refers to the amounts of an inhibitor of FtsZ and a β-lactam antibiotic, in combination, which result in the restoration of the susceptibility of a methicillin-resistant Staphylococcal bacteria to a β-lactam antibiotic. In therapeutic applications, the methods and compositions of the invention are used for administration to a patient already suffering from an infection from bacteria, in an amount sufficient to cure or at least partially arrest the symptoms of the infection. Amounts effective for this use will depend on the severity and course of the infection, previous therapy, the patient's health status and response to the drugs, and the judgment of the treating physician. Therapeutically effective amounts can be assessed by clinical trial results and/or model animal infection studies.

The organisms amenable to therapy by way of the various combinations disclosed herein include methicillin-resistant S. aureus (MRSA) and methicillin-resistant S. epidermis (MRSE) and other coagulase negative Staphylococci (MRCNS) including Staphylococcus haemolyticus, Staphylococcus saprophyticus, and Staphylococcus carnosus.

Bacterial infections treatable with the methods and compositions of the invention include, but are not limited to, complicated intra-abdominal infection, appendicitis, acute pelvic infections, complicated urinary tract infections, complicated skin and skin structure infections, diabetic foot ulcer, community-acquired pneumonia, nosocomial pneumonia, acute pulmonary excerbations in cystic fibrosis patients, febrile neutropenia, lower respiratory infections, bacterial septicemia, bone and joint infection, endocarditis, polymicrobial infection, and bacterial meningitidis. See Zhanel et al., 2007, Drugs 67:10274052 and Dalhoff et al., 2006, Biochem Pharmacol 71:1085-1095.

Pharmaceutical Compositions

The inhibitor of FtsZ and/or β-lactam may be in a pharmaceutical composition containing the inhibitor of FtsZ and/or β-lactam and a pharmaceutically acceptable carrier or excipient. In certain embodiments of the invention, a pharmaceutical composition comprises, or consists essentially of, an inhibitor of FtsZ, a β-lactam, and a pharmaceutically acceptable carrier or excipient. The inhibitor of FtsZ and β-lactam are in such amounts and relative proportion that the combination constitutes a pharmaceutically or therapeutically effective dose or amount. The compounds can be prepared as pharmaceutically acceptable salts (i.e., non-toxic salts which do not prevent the compound from exerting its effect). The β-lactam antibiotic may be a carbapenem, cephalosporin/cephamycin, monolactam or penicillin.

In certain embodiments, a composition of the invention further comprises a β-lactamase inhibitor.

Pharmaceutically acceptable carriers or excipients can be used to facilitate administration of the compound, for example, to increase the solubility of the compound. Solid carriers include, e.g., starch, lactose, dicalcium phosphate, microcrystalline cellulose, sucrose, and kaolin, and optionally other therapeutic ingredients. Liquid carriers include, e.g., sterile water, saline, buffers, polyethylene glycols, non-ionic surfactants, and edible oils such as corn, peanut and sesame oils, and other compounds described e.g., in the MERCK INDEX, Merck & Co., Rahway, N.J. In addition, various adjuvants such as are commonly used in the art may be included. For example: flavoring agents, coloring agents, preservatives, and antioxidants, e.g., vitamin E, ascorbic acid, BHT and BHA. Various other considerations are described, e.g., in Gilman et al. (eds) (1990) Goodman and Gilman's. The Pharmacological Basis of Therapeutics, 8th Ed., Pergamon Press. Methods for administration are discussed therein, e.g., for oral, sublingual, intravenous, intraperitoneal, or intramuscular administration, subcutaneous, topically, and others.

The pharmaceutical compositions described herein may be presented in a number of appropriate dosage forms; e.g., tablets, capsules, pills, powders, suspensions, solutions, and the like, for oral administration; solutions, suspensions, emulsions, and the like, for parenteral administration; solutions for intravenous administration; and ointments, transdermal patches, and the like, for topical administration. The preferred form depends on the intended mode of administration and therapeutic application. For some compounds a pharmacologically acceptable salt of the compound will be used to simplify preparation of the composition. The compounds may be employed in powder or crystalline form, in liquid solution, or in suspension. The compounds described herein may be prepared in crystalline form by for example dissolution of the compound in water, preferably in the minimum quantity thereof, followed by admixing of this aqueous solution with a water miscible organic solvent such as a lower aliphatic ketone such as a di-(C₁₋₆) alkyl ketone, or a (C₁₋₆) alcohol, such as acetone or ethanol.

Compositions for injection, a preferred route of delivery, may be prepared in unit dosage form in ampules, or in multidose containers. The injectable compositions may take such forms as suspensions, solutions, or emulsions in oily or aqueous vehicles, and may contain various formulating agents. Alternatively, the active ingredient may be in powder (lyophillized or non-lyophillized) form for reconstitution at the time of delivery with a suitable vehicle, such as sterile water. In injectable compositions, the carrier is typically comprised of sterile water, saline or another injectable liquid, e.g., peanut oil for intramuscular injections. Also, various buffering agents, preservatives and the like can be included.

Topical applications may be formulated in carriers such as hydrophobic or hydrophilic bases to form ointments, creams, lotions, in aqueous, oleaginous or alcoholic liquids to form paints or in dry diluents to form powders.

Oral compositions may take such forms as tablets, capsules, oral suspensions and oral solutions. The oral compositions may utilize carriers such as conventional formulating agents, and may include sustained release properties as well as rapid delivery forms.

Compositions intended for oral use may be prepared according to methods known to the art for the manufacture of pharmaceutical compositions and such compositions may contain one or more agents selected from the group consisting of sweetening agents, flavoring agents, coloring agents and preserving agents in order to provide pharmaceutically elegant and palatable preparation. See, e.g., Remington: The Science and Practice of Pharmacy, 21^{st} ed., Lippincott Williams & Wilkins, 2005. Tablets containing the active ingredient in admixture with non-toxic pharmaceutically acceptable excipients may also be manufactured by known methods. The excipients used may be for example, (1) inert diluents such as calcium carbonate, sodium carbonate, lactose, calcium phosphate or sodium phosphate; (2) granulating and disintegrating agents such as corn starch, or alginic acid; (3) binding agents such as starch, gelatin or acacia, and (4) lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in U.S. Pat. Nos. 4,256,108; 4,160,452; and 4,265,874 to form osmotic therapeutic tablets for controlled release.

In some cases, formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, for example peanut oil, liquid paraffin, or olive oil.

Aqueous suspensions normally contain the active materials in admixture with excipients suitable for the manufacture of aqueous suspensions. Such excipients may be suspending agents such as sodium carboxymethylcellulose, methylcellulose, hydroxypropylmethylcellulose, sodium alginate, polyvinylpyrrolidone, gum tragacanth and gum acacia; dispersing or wetting agents which may be a naturally-occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived from a fatty acid and a hexitol such as polyoxyethylene sorbitol monooleate, or a condensation product of ethylene oxide with a partial ester derived from a fatty acid and a hexitol anhydride, for example polyoxyethylene sorbitan monooleate.

The aqueous suspensions may also contain one or more preservatives, for example, ethyl or n-propyl p-hydroxybenzoate; one or more coloring agents; one or more flavoring agents; and one or more sweetening agents such as sucrose or saccharin.

Dispersible powders and granules are suitable for the preparation of an aqueous suspension. They provide the active ingredient in admixture with a dispersing or wetting agent, a suspending agent and one or more preservatives. Suitable dispersing or wetting agents and suspending agents are exemplified by those already mentioned above. Additional excipients, for example, those sweetening, flavoring and coloring agents described above may also be present.

An oily suspension may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be preserved by the addition of an antioxidant such as ascorbic acid.

The pharmaceutical compositions of the invention may also be in the form of oil-in-water emulsions. The oily phase may be a vegetable oil such as olive oil or arachis oils, or a mineral oil such as liquid paraffin or a mixture thereof. Suitable emulsifying agents may be (1) naturally-occurring gums such as gum acacia and gum tragacanth, (2) naturally-occurring phosphatides such as soy bean and lecithin, (3) esters or partial esters derived from fatty acids and hexitol anhydrides, for example, sorbitan monooleate, (4) condensation products of said partial esters with ethylene oxide, for example, polyoxyethylene sorbitan monooleate. The emulsions may also contain sweetening and flavoring agents.

Syrups and elixirs may be formulated with sweetening agents, for example, glycerol, propylene glycol, sorbitol or sucrose. Such formulations may also contain a demulcent, a preservative and flavoring and coloring agents.

When a carbapenem is used, the pharmaceutical compositions described above may be combined or used with dehydropeptidase (DHP) inhibitors. Many carbapenems are susceptible to attack by the renal enzyme DHP. This attack or degradation may reduce the efficacy of the carbapenem. Many carbapenems, on the other hand, are less subject to such attack, and therefore may not require the use of a DHP inhibitor. However, such use is optional and contemplated to be part of the present invention. Inhibitors of DHP and their use with carbapenems are disclosed in, e.g., U.S. Pat. No. 5,071,843 and European Patent Nos. EP 0 007 614 and EP 0 072 014. When a DHP inhibitor is used with a pharmaceutical invention described above, the DHP inhibitor may be in a pharmaceutical composition with a pharmaceutically acceptable carrier or excipient. A preferred DHP inhibitor is 7-(L-2-amino-2-carboxyethylthio)-2-(2,2-dimethylcyclopropanecarboxamide)-2-heptenoic acid, also known as cilastin, or a useful salt thereof.

In one aspect of the invention, the combination of the DHP inhibitor and the carbapenem can be in the form of a pharmaceutical composition containing the two compounds in a pharmaceutically acceptable carrier. The two can be employed in amounts so that the weight ratio of the penem to inhibitor is 1:3 to 30:1 or 1:1 to 5:1. An exemplary weight ratio of carbapenem:DHP inhibitor in the combination compositions is about 1:1.

In another aspect of the invention, pharmaceutical compositions of the present invention contemplate an inhibitor of FtsZ in combination with a carbapenem, a DHP inhibitor such as, cilastatin, and a pharmaceutically acceptable carrier.

Dosage and Administration

The pharmaceutical compositions of the invention can be administered parenterally (intravenously or intramuscularly), or subcutaneously, particularly when they are used in combination with a β-lactam antibiotic or comprise a β-lactam antibiotic. They may also be administered orally or sublingually. The compounds of this invention may also be used to treat topical antibacterial infection.

The amount of active ingredients that may be combined with the carrier materials to produce a single dosage form will vary depending upon the host treated and the particular mode of administration.

In the various embodiments of the invention, the β-lactam antibiotic and inhibitor of FtsZ can be administered sequentially or concurrently. Preferably, the β-lactam antibiotic and inhibitor of FtsZ are administered together. When administered concurrently, the β-lactam antibiotic and inhibitor of FtsZ may be administered in the same formulation or in separate formulations. When administered sequentially, either the β-lactam or inhibitor of FtsZ may be administered first. After administration of the first compound, the other compound is administered preferably within 1, 2, 3, 4, 5, 10, 15, 20, 25, 30, or 60 minutes. In one aspect of the invention, when a DHP inhibitor is used with a carbapenem, it may be administered separately, or in a formulation with a potentiator and/or carbapenem.

Exemplary intravenous or intramuscular dosages of an inhibitor of FtsZ are in the range of 50 mg to 2 g, 100 mg to 1 g, 250 mg to 750 mg. Other exemplary dosages for intravenous or intramuscular use include ranges of 0.1 to 200, 0.2 to 100, 0.5 to 50, or 1 to 25 mg/kg/day. Preferably, the dosage is given 1, 2, 3 or 4 times daily.

β-lactam antibiotics may suitably be administered to the patient at a daily dosage of from 0.1 to 200, 0.2 to 100, 0.5 to 75, 0.7 to 50, 1 to 25, or 5 to 20 mg/kg of body weight. About 5 to 50 mg of a β-lactam per kg of body weight is preferred. Preferably, the dosage is given 1, 2, 3 or 4 times daily. For instance, the β-lactam can be administered intramuscularly or intravenously in amounts of 1-100 mg/kg/day, preferably 1-20 mg/kg/day, or 1-5 mg/kg/dose, in divided dosage forms, e.g., 1, 2, 3 or 4 times daily.

For an adult human (of approximately 70 kg body weight), from 50 to 3000 mg, preferably from 100 to 1000 mg, of β-lactam may be administered daily, suitably in from 1, 2, 3, 4, 5, or 6 separate doses. When the β-lactam is presented in unit dosage form, each unit dose may suitably comprise from about 25 to about 1000 mg, preferably about from 50 to about 500 mg, of β-lactam. Each unit dose may, for example, be 62.5, 100, 125, 150, 200 or 250 mg of a β-lactam. The preferred dosage is 250 mg to 1000 mg of the antibacterial given one to four times per day. More specifically, for mild infections a dose of about 250 mg two or three times daily is recommended. For moderate infections against highly susceptible gram positive organisms a dose of about 500 mg three or four times daily is recommended. For severe, life-threatening infections against organisms at the upper limits of sensitivity to the antibiotic, a dose of about 1000-2000 mg three to four times daily may be recommended.

For children, a dose of about 1 to 100, 2.5 to 50, 5 to 25, or 10 to 20 mg/kg of body weight is preferred; a dose of 10 mg/kg is typically recommended. Preferably the dosage is given 1, 2, 3, or 4 times per day. Unit dosages may be as used for adults.

The compositions for human delivery per unit dosage may contain from about 0.01% to as high as about 99% of active material, the preferred range being from about 10-60%. The composition will generally contain from about 15 mg to about 2.5 g of the active ingredient; however, in general, it is preferable to employ dosage amounts in the range of from about 250 mg to 1000 mg. In parenteral administration, the unit dosage will typically include the pure compound in sterile water solution or in the form of a soluble powder intended for solution, which can be adjusted to neutral pH and isotonic.

When the inhibitor of FtsZ is co-administered with a β-lactam, the ratio of the compound of the inhibitor of FtsZ to β-lactam may vary within a wide range. The ratio may, for example, be from 100:1 to 1:100; more particularly, it may, for example, be from 2:1 to 1:30. The amount of β-lactam according to the invention will normally be approximately similar to the amount in which it is conventionally used.

The dosage to be administered depends to a large extent upon the condition and size of the subject being treated, the route and frequency of administration, the sensitivity of the pathogen to the particular compound selected, the virulence of the infection and other factors. Such matters, however, are left to the routine discretion of the physician according to principles of treatment well known in the antibacterial arts. Another factor influencing the precise dosage regimen, apart from the nature of the infection and peculiar identity of the individual being treated, is the molecular weight of the compound.

In embodiments where a β-lactamase inhibitor is used, the molar β-lactam antibiotic to β-lactamase inhibitor ratio is from 2:1 to 18:1, preferably from 2:1 to 4:1.

In one aspect of the invention, a DHP inhibitor is also administered either sequentially or concurrently from the inhibitor of FtsZ and/or carbapenem. The DHP inhibitor can be administered, orally, intramuscularly, or IV, in amounts of 1-100 mg/kg/day, or preferably 1-30 mg/kg/day, or 1-5 mg/kg/dose and may be in divided dosage forms, e.g., three or four times a day.

The specific embodiments described herein are offered by way of example only, and the invention is to be limited only by the terms of the appended claims along with the full scope of equivalents to which such claims are entitled. Indeed various modifications of the invention, in addition to those shown and described herein will become apparent to those skilled in the art from the foregoing description and accompanying drawings. Such modifications are intended to fall within the scope of the appended claims.

### EXAMPLES

### Example 1

### Genetic Validation of FTSZ

Genetic validation that inhibition of FtsZ, FtsA or FtsW restores MRSA susceptibility to general β-lactams, including cephalosporins and carbapenems, was achieved in MRSA strain COL (Merck Culture Collection, Rahway, N.J.) by transforming plasmid pEPSA5 bearing xylose-regulated antisense fragments corresponding to FtsZ (SACOL1199), FtsA (SACOL1198), or FtsW (SACOL2075). See Forsyth et al., 2002, Mol. Microbial 43:1387-1400 for details concerning plasmid and antisense interference construction. Strains were grown overnight in Luria Bertani (LB) Miller broth at 37° C. containing 34 μg/ml chloramphenicol. Assay plates were prepared by seeding 10⁷ cells/ml of each culture into 48° C. cooled LB Miller agar containing 34 μg/ml chloramphenicol. To partially repress FtsZ expression, 50 mM xylose was supplemented to plates. This antisense inducer concentration modestly impairs growth of each strain equally, reflecting a 20% inhibitory growth concentration (i.e., xylose IC₂₀˜50 mM).

The following antibiotics were then spotted on the surface of the plates: carbapenems, imipenem (12.8 μg) and ertapenem (51.2 μg); cephalosporins, cefepime (102.4 μg), ceftazidime (102.4 μg), and ceftriaxone (102.4 μg); and the β-lactam piperacillin (combined with the β-lactamase tazobactam in a 8:1 molar ratio (25.6 μg). As a control, MRSA COL was identically tested but under conditions where no xylose was supplemented into the medium (i.e., non-repressed FtsZ) as well as by identically processing the MRSA COL strain transformed with pEPSA5 but lacking an antisense fragment (data not shown). As a control for the specificity of ftsZ, ftsA, and ftsW mutant susceptibility to β-lactams, the non-β-lactam cell wall antibiotic, vancomycin (0.8 μg) was additionally spotted. Plates were then incubated at 37° C. for 16 h and photographed.

The effect FtsZ, FtsA, or FtsW imparts on MRSA susceptibility to the above antibiotics was determined by comparing their zone of inhibition between plates supplemented with antisense inducer (50 mM xylose) versus those without xylose. Differential antibiotic zones of inhibition between FtsZ, FtsA, and FtsW antisense bearing strains grown under xylose induced versus non-induced conditions reflect Fts gene-dependent restoration of MRSA COL susceptibility to β-lactams.

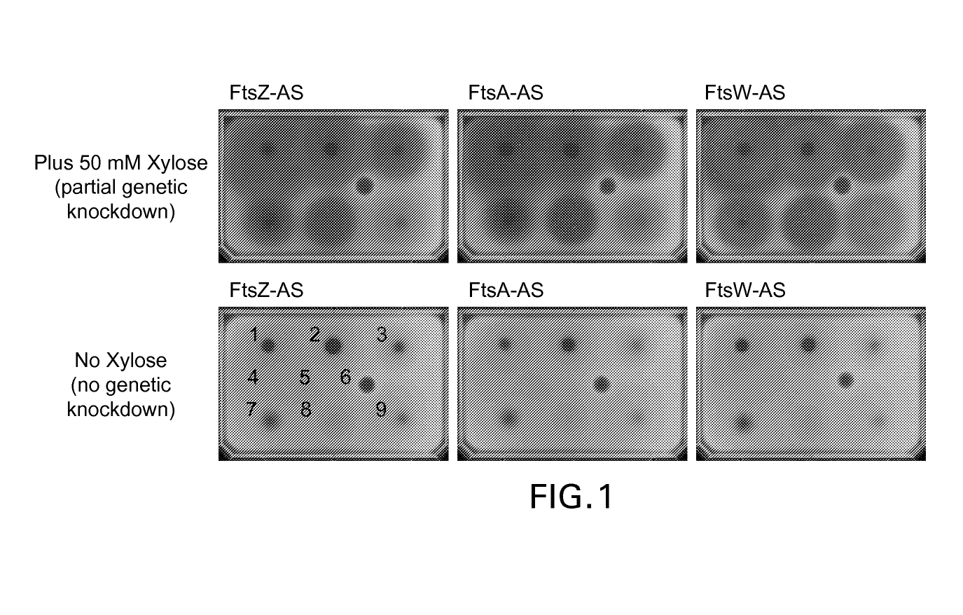

As shown in FIG. 1, partial genetic inactivation of FtsZ, FtsA, or FtsW resulted in marked hypersensitivity to all of the above β-lactams, cephalosporins, and carbapenems tested. Conversely, none of the FtsZ-AS, FtsA-AS, or FtsW-AS strains demonstrated altered hypersensitivity to vancomycin or other mechanistically distinct antibiotics, including fluorquinolones (DNA gyrase inhibitors; ciprofloxacin, levofloxacin), trimethoprim (folate biosynthesis), rifampicin (RNA polymerase), protein synthesis inhibitors (tetracycline, linezolid, azithromycin), or fatty acid biosynthesis (cerulenin) (data not shown). Zone sizes for parallel spottings on FtsZ-AS, FtsA-AS, and FtsW-AS inoculated media lacking xylose were identical to MRSA COL maintaining the vector control, pEPSA5 grown in the presence or absence of 50 mM xylose supplement (data not shown).

Collectively, these data demonstrate genetic inactivation of FtsZ, as well as additional components of the Z ring cytoskeleton, specifically enhance the activity of β-lactams, cephalosporins, and carbapenems against MRSA. Importantly, these genetic data predict that target-specific inhibitors of FtsZ would similarly hypersensitize MRSA to β-lactam class antibiotics, thus restoring their activity against said drug-resistant Staphyococci.

### Example 2

### Pharmacological Validation of FTSZ

To demonstrate restoration of the activity of β-lactam antibiotics against methicillin-resistant Staphylococci, the known FtsZ inhibitor, PC190723 (See Haydon et al., 2008, Science 321:1673-1675), was first evaluated by a complete checkerboard assay in which a full dilution series of PC190723 was tested in combination across a full dilution series of imipenem and ertapenem for potential synergistic effects against MRSA and MRSE.

The protocols used to pharmacologically demonstrate that FtsZ inhibitors synergize in combination with β-lactams in vitro are described below.

### Materials:

### Agents:

Imipenem (Merck, Whitehouse Station, N.J.): stock solutions prepared in 10 mM MOPS buffer, pH 7
Ertapenem (Merck, Whitehouse Station, N.J.): solutions prepared in 10 mM MOPS buffer, pH 7
Cefazolin: from United States Pharmacopeia (USP), stock solutions prepared in DMSO.
Cefdinir: from USP, stock solutions prepared in DMSO
Cefepime: from USP, stock solutions prepared in DMSO
Ceftazidime: from Sigma Aldrich, stock solutions prepared in 0.1 N NaOH
Amoxicillin: from USP, stock solutions prepared in 50% DMSO

### Bacterial Strains: (Merck Culture Collection, Rahway, N.J.)

COL: Methicillin-resistant S. aureus
MB6255 (CLB26329) Methicillin-resistant S. epidermidis

### Microbiological Growth Media:

Cation adjusted Mueller-Hinton broth (CAMHB): BD BBL (Fisher Scientific)
Trypticase Soy Broth: (TSB; Soybean-Casein digest medium); Bacto BD (Fisher Scientific)

### Media Preparation

Prepared according to manufacturer's instructions (22 g (CAMHB) or 30 g (TSB) dissolved in 1000 mL water; autoclaved 22 minutes). Stored refrigerated. Filter-sterilized before use using a Coming 0.45 Tm cellulose acetate filter.

### Checkerboard Studies

The checkerboard method is the technique most frequently used to assess antimicrobial combinations in vitro. See Antibiotics in Laboratory Medicine Victor Lorian ed. 2005. The term. checkerboard refers to the pattern of microtiter wells formed by multiple dilutions of the two antimicrobials being tested in concentrations equal to, above and below their minimal inhibitory concentrations (MICs) against the organisms being tested. The checkerboard consists of columns in which each well contains the same amount of Drug A being two-fold diluted along the x-axis and rows in which each well contains the same amount of Drug B being two-fold diluted on the Y-axis. The result is that each well contains a unique combination of the two drugs being tested. Also tested is the antimicrobial activity of each agent singly.

The 96 well U-bottom polypropylene microtiter plates (Fisher Scientific, Pittsburgh, Pa.) were inoculated with 90 μl of bacterial cells (grown in Tryptic Soy Broth at 37° C. for 18 hours with rotary shaking) diluted in cation-adjusted Mueller Hinton broth to a final concentration of ˜5.5×10⁵ CFU/mL.

β-lactam antibiotics were two-fold serially diluted along the Y-axis of 96-well U-bottom microtiter dishes (Fisher Scientific, Pittsburgh, Pa.) in microbiological growth media [brain heart infusion broth (BBL)]. FtsZ inhibitors were serially diluted along the X-axis of 96-well U-bottom microtiter dishes in microbiological growth media. The plates were inoculated with bacterial cells (grown in Trypticase Soy Broth at 37° C. for 18 hours with rotary shaking) to a final concentration of ˜5×10⁵ CFU/mL. Test plates were incubated stationery at 37° C. for 22-24 hours. Minimum Inhibitory Concentrations (MICs) of each agent were defined as the minimum concentration of agent necessary to completely inhibit visible growth.

The FIC index is most commonly used to report results of studies with antimicrobial combinations. In this method, the FIC for each drug is derived by dividing the concentration of that drug necessary to inhibit growth in a given combination by the MIC of the test organism for that drug alone. The FTC index (FICI) is then calculated by summing the separate FICs of the drugs present in that test well. Synergism is defined as an FTC index of 0.5.

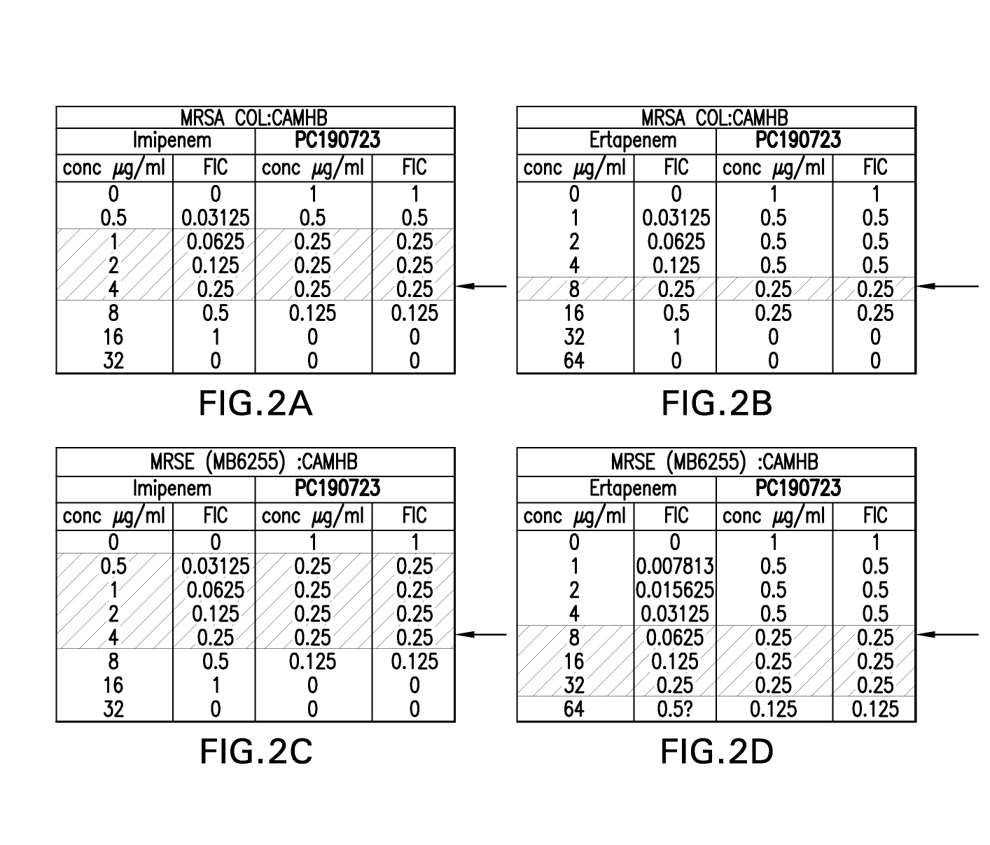

The results are shown in FIG. 2. Consistent with the highly conserved nature of FtsZ across Staphylococci, PC190723 displayed the same minimal inhibitory concentration (MIC) of 1.0 μg/ml across MRSA and MRSE. Importantly, as predicted by genetic potentiation studies between FtsZ antisense knock down and resulting hypersensitivity towards β-lactam antibiotics, PC190723 demonstrated significant synergy (FIC=0.25) against MRSA and MRSE in combination with either imipenem (FICI=0.31) or ertapenem (FICI=0.5) at their respective clinical breakpoints (4 μg/ml and 8 μg/ml, respectively).

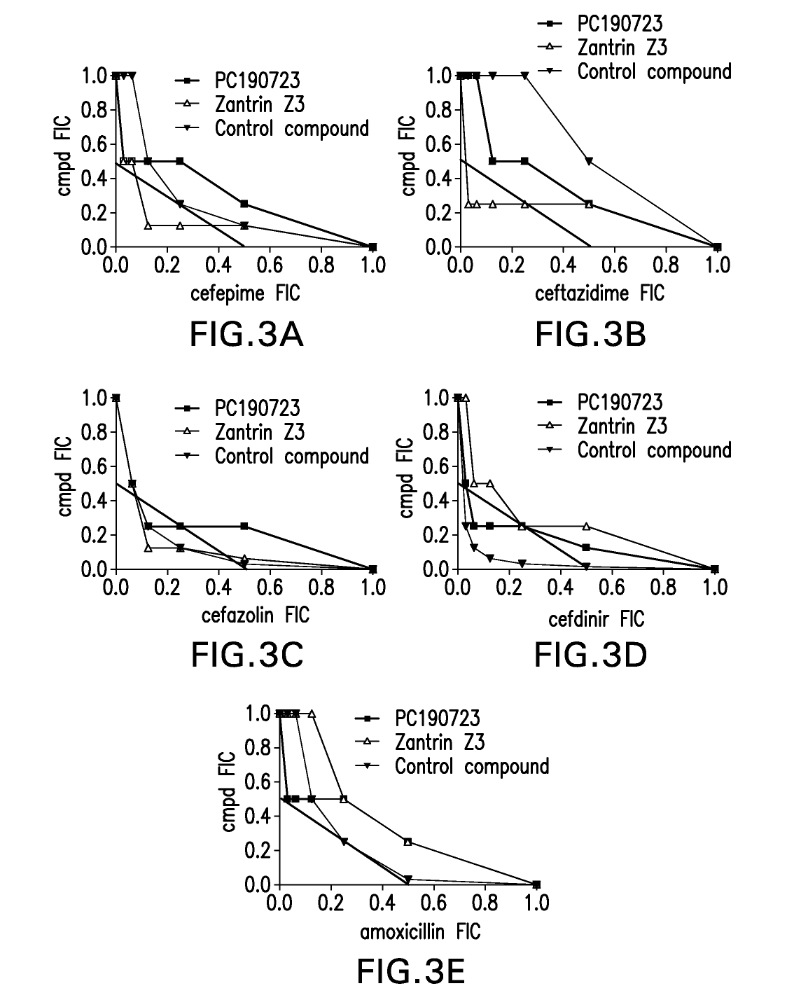

To further demonstrate antibacterial synergy between FtsZ inhibitors and β-lactam antibiotics against MRSA, additional checkerboard assays were performed using PC190723 (See Haydon et al., 2008, Science 321:1673-1675) and zantrin Z3 (Margalit, 2004, Proc Natl Acad Sci USA 101:11821-11826) versus a broad set of cephalosporins (cefipime, ceftazidime, cefazolin, and cefdinir), as well as the penicillin, amoxicillin. FTC indexes of paired agents are plotted as isobolograms (FIGS. 3A-E).

All FTC indexes less than or equal FICI 0.5 reflect the statistically significant synergy and are highlighted. PC190723 demonstrated significant synergy at FIC=0.25 in combination with a subset of cephalosporins tested, including cefazolin (FICI=0.38) and cefdinir (FICI=0.31), an orally administered cephalosporin. Similarly, PC190723 augments the activity of amoxicillin (FICI=0.53), reducing the effective inhibitory concentration of the amoxicillin ˜32-fold when combined with PC190723 at FIC=0.5. Zantrin Z3 at FTC values ranging between 0.125 and 0.25 also potentiated the effects of β-lactam antibiotics, demonstrating significant synergy in combination with cefipime (FICI=0.25), ceftazidime (FICI=0.28), cefazolin (FICI=0.25), and cefdinir (FICI=0.5), although no synergy was detected in combination with amoxicillin.

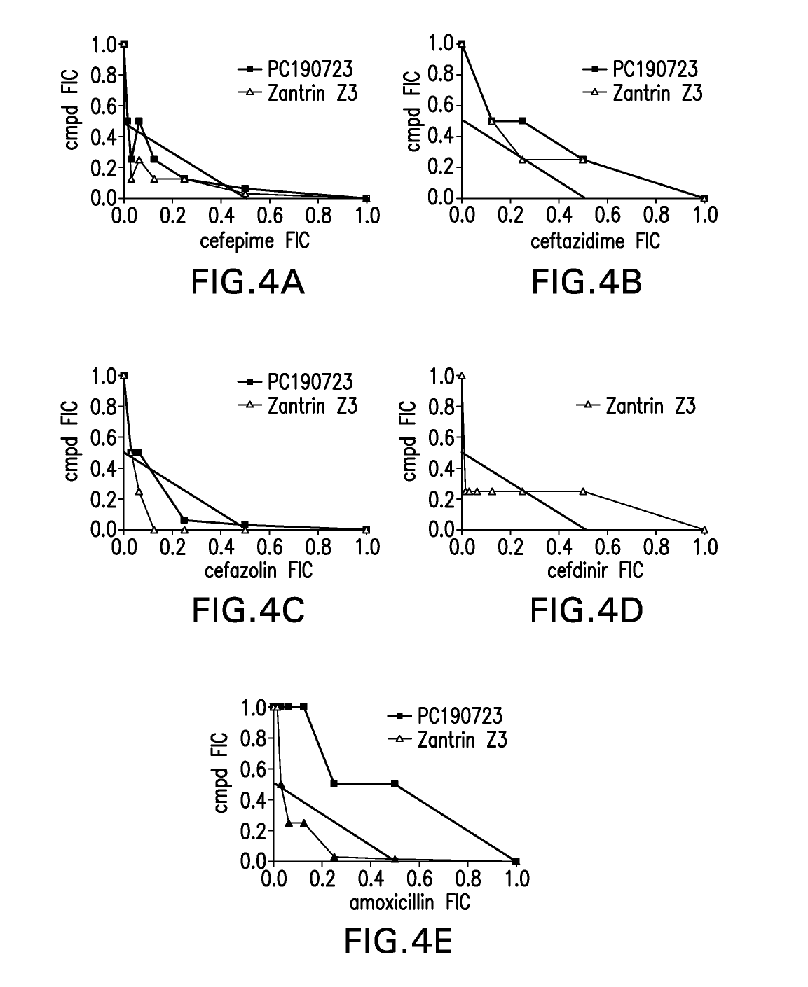

Similarly, checkerboard assays were performed to demonstrate antibacterial synergy against MRSE using PC190723 and zantrin Z3 in combination with the above and β-lactam antibiotics (FIGS. 4A-E).

PC190723 demonstrated significant synergy against MRSE when combined with cefepime, with the lowest achievable FICI=0.28 for this drug pairing. PC190723 also demonstrated synergy in combination with ceftazolin (FICI=0.31). Zantrin Z3 at FIC values ranging between 0.031 and 0.25 markedly potentiated the effects of all β-lactam antibiotics tested, with the following FIC indexes; cefipime (FICI=0.16), ceftazidime (FICI=0.50), cefazolin (FICI=0.31), cefdinir (FICI=0.27), and amoxicillin (FICI=0.28).

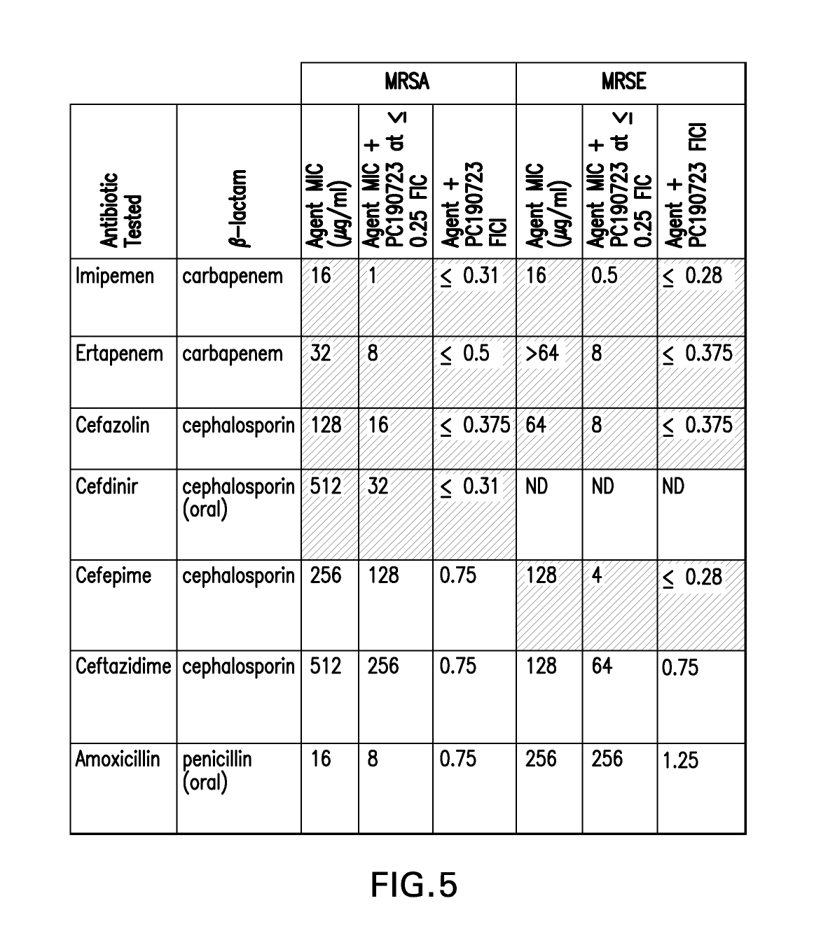

Collectively, in vitro checkerboard assay results presented in EXAMPLE 2 provide rigorous pharmacological validation that structurally diverse FtsZ inhibitors potentiate the activity of β-lactam antibiotics against MRSA as predicted by genetic validation studies in EXAMPLE 1 using antisense-mediated knockdown of Fts activity (FIG. 1). Pharmacological validation of in vitro synergy between these FtsZ inhibitors and a broad set of β-lactam antibiotics is also extended to MRSE. Importantly, by pairing PC190723 in combination with imipenem or ertapenem, we demonstrate that either carbepenem regains activity against MRSA and MRSE at or below their clinical breakpoint to these methicillin-resistant Staphalococci (FIG. 5).

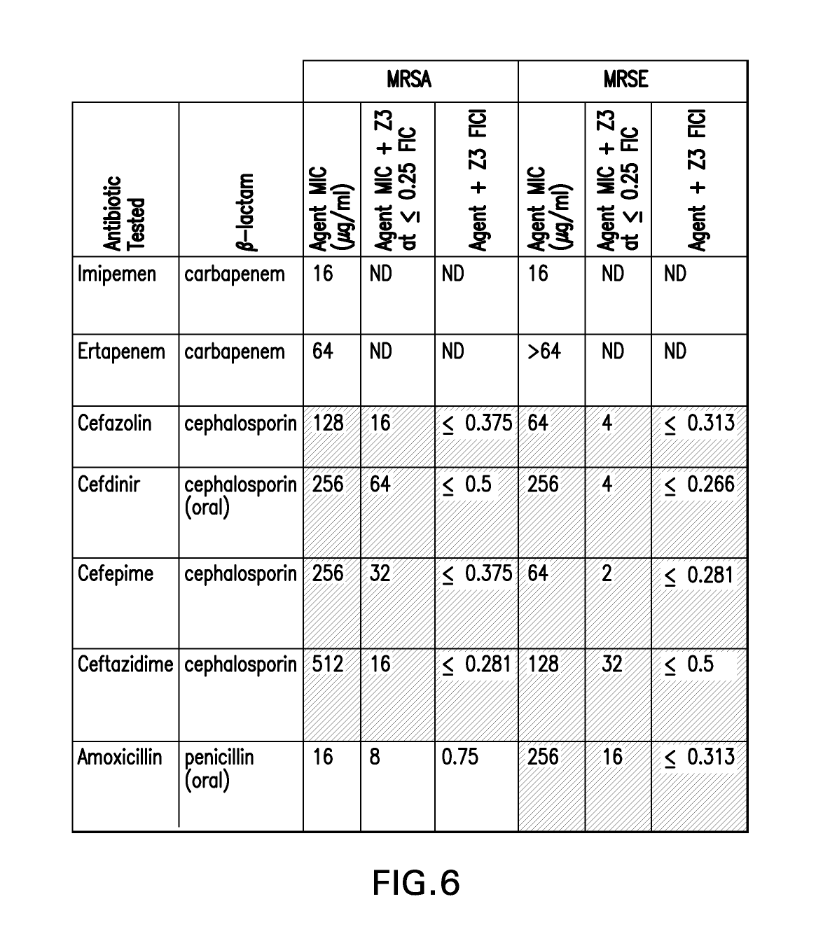

These findings provide a dual strategy of both restoring β-lactam antibiotic efficacy against said drug resistant bacteria while requiring significantly reduced levels of the β-lactam antibiotic. For example, we demonstrate PC190723 (See Haydon et al., 2008, Science 321:1673-1675) at sub-inhibitory drug concentrations (FIC≦0.25) when combined with imipenem, results in 16-fold and 32-fold lower levels of the carbapenem required to inhibit growth of MRSA and MRSE, respectively (FIG. 5). Additional potentiation effects of PC190723 in combination with cephalosporins and ertapenem are summarized in FIG. 6. Similarly, we demonstrate zantrin Z3 at FIC≦0.25 when combined with the cefazolin, results in 16-fold lower levels of this cephalosporin required to inhibit growth of MRSA or MRSE (FIG. 6. Additional potentiation effects of zantrin Z3 in combination with β-lactams are summarized in FIG. 6. Most notably the cephalosporin, cefdinir, which if paired with Z3 requires a 64-fold reduced drag concentration to restore anti-MRSE activity, and amoxicillin requiring 1/16^{th} its normal drug concentration to display MRSE efficacy (FIG. 6).

### Example 3

### PO Dosing in MRSA Deep Thigh Model to Demonstrate In Vivo Synergy and Efficacy

Female mice (20-25 g) were rendered neutropenic via intraperitoneal injection of cyclophosphamide (Mead Johnson Pharmaceuticals) four days (150 mg/kg) and one day (100 mg/kg) prior to experimental infection. Neutropenic mice were infected on Day 0 via intramuscular thigh injection of 0.1 ml containing ˜1-2×10⁶ CPU Staphylococcus aureus (strain B, MRSA COL) from freshly grown culture broth. Two hours post thigh infection, mice were randomized and groups were administered increasing doses of PC190723 (50, 100, 200 mg/kg orally (p.o.), t.i.d (q3 hr); equivalent to 150, 300, and 600 mg/kg/24 hr) in the absence or presence of imipenem/cilastatin (10 mg/kg/50 mg/kg subcutaneously (s.c.), t.i.d. (q3 hr); equivalent to 30 mg/kg/24 hr and 150 mg/ kg/24 hr). Assay control groups included non-infected, vehicle-treated mice, vehicle-treated baseline infected (t=0 hr) mice, vehicle-treated 24 hr infected (t=24 hr) mice, and linezolid ((Zyvox IV solution, Bell Medical; 40 mg/kg orally (p.o.), b.i.d. (q6 hr) equivalent to 80 mg/kg/24 hr).

### Groups:

Group 1: Vehicle (no innoculum) n=3

Group 2: Vehicle (innoculum) T=0 n=4

Group 3: Vehicle (innoculum) T=24 n=4

Group 4: Imipenem 10 mpk (t.i.d., s.c.) n=4

Group 5: PC190723 50 mpk (t.i.d., p.o.) n=4

Group 6: PC190723 100 mpk (t.i.d., p.o.) n=4

Group 7: PC190723 200 mpk (t.i.d., p.o.) n=4

Group 8: Imipenem 10 mpk (t.i.d., s.c.)+PC190723 50 mpk (t.i.d., p.o.) n=4

Group 9: Imipenem 10 mpk (t.i.d., s.c.)+PC190723 100 mpk (t.i.d., p.o.) n=4

Group 10: Imipenem 10 mpk (t.i.d., s.c.)+PC190723 200 mpk (t.i.d., p.o.) n=4

Group 11: Linezolid 40 mpk (b.i.d., p.o.) n=4

Twenty four hours post the initiation of therapy, mice were sacrificed, thighs aseptically removed, placed in 4 ml sterile phosphate buffered saline (Fisher Scientific), and homogenized using a Polytron (Brinkmann Instruments). Homogenates were serially 100-fold diluted in 9.9 ml sterile saline and plated on Mannitol Salt Agar plates. Plates were incubated at 35° C. for 48 hours and colony forming units (CFU) of bacteria remaining per thigh were determined.

### Results

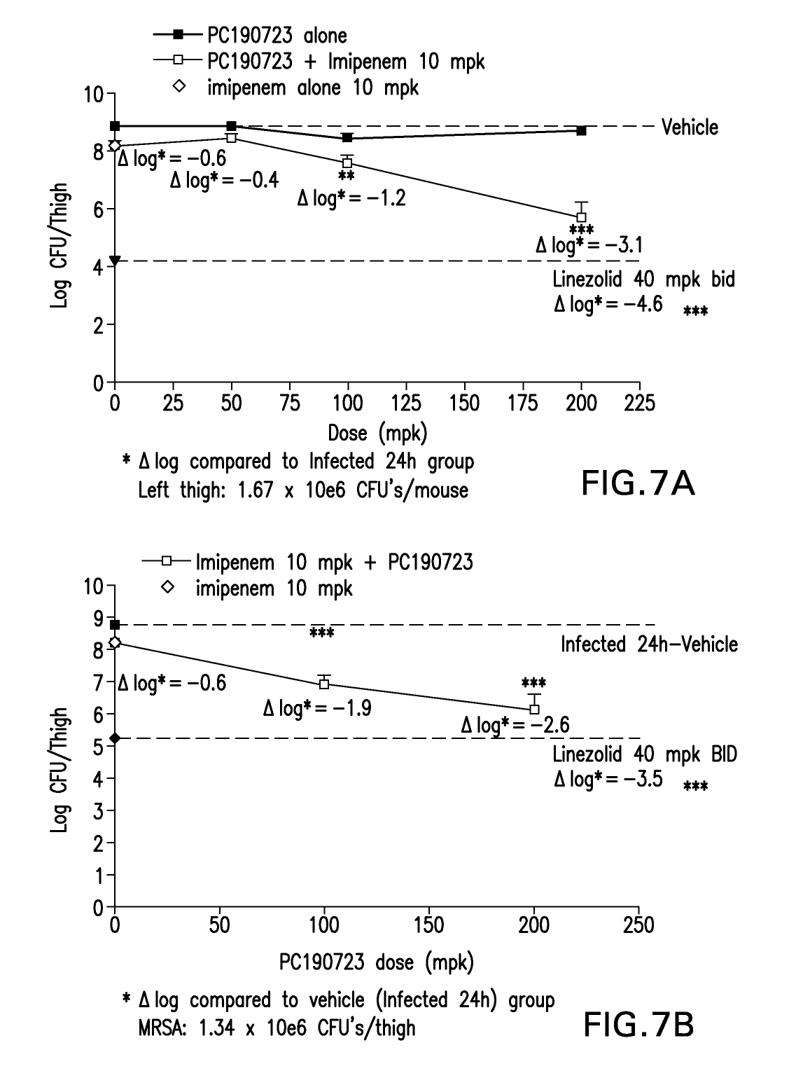

At the start of therapy mice had 5.3 Log 10 CFU of MRSA COL/thigh (study 1, 4A) and 5.6 Log 10 CFU of MRSA COL/thigh (Study 2, 4B). After 24 h the organisms grew to 8.8 log 10 CFU/thigh (FIGS. 7A and 7B) in saline-treated control mice. Imipenem or increasing doses of PC190723 administered as monotherapies did not significantly alter the MRSA COL bacterial burden in the thighs over 24 hr therapy. In contrast, significant and dose-dependent antibacterial synergy was observed when individual non-efficacious doses of imipenem and PC190723 were combined. Combination therapy over 24 hr resulted in −0.4, −1.2, −3.1 Log 10 CFU (FIG. 7A) reduction at the 50, 100, 200 mg/kg doses and −1.9, −2.6 Log 10 CFU (FIG. 7B) reduction at the 100 and 200 mg/kg doses, respectively, with the 10 mg/kg imipenem+200 mg/kg PC190723 approaching linezolid-like antibacterial activity. The findings demonstrate that combining individually nonefficacious doses of impenem and PC190723 synergizes to reduce MRSA COL infection in vivo in a robust and reproducible manner.

In conclusion, EXAMPLES 1-3 provide rigorous genetic and pharmacological demonstration in vitro and in vivo that FtsZ inhibitors at sub-efficacious doses can effectively restore susceptibility of methicillin-resistant Staphylococci to β-lactam antibiotics. Further, the synergistic effects between these antibiotic drug classes provides efficacy in an animal model of MRSA infection where neither agent alone is sufficient to achieve this effect. Thus, FtsZ inhibitors when paired with carbapenem, cephalosporin, or penicillin β-lactam antibiotics provides a novel combination therapy strategy to treating a bacterial infection of methicillin-resistant Staphylococci.

1. A method of treating a bacterial infection of methicillin-resistant Staphylococci in a mammalian patient in need of such treatment comprising administering to said patient an effective amount of an inhibitor of FtsZ in combination with an effective amount of a β-lactam antibiotic.

2. The method of claim 1, wherein the β-lactam antibiotic is a carbapenem antibiotic.

3. The method of claim 2, wherein the carbapenem antibiotic is imipenem or ertapenem.

4. The method of claim 1 which further comprises administering a β-lactamase inhibitor.

5. (canceled)

6. (canceled)

7. The method of claim 1, wherein the methicillin-resistant Staphylococci are Staphylococcus aureus or Staphylococcus epidermidis.

8. The method of claim 1, wherein the patient is a human.

9. The method of claim 1, wherein the FtsZ inhibitor and β-lactam antibiotic are administered concurrently.

10. The method of claim 9, wherein the FtsZ inhibitor and β-lactam antibiotic are administered in the same formulation.

11. The method of claim 1, wherein the FtsZ inhibitor and β-lactam antibiotic are administered sequentially.

12. The method of claim 11, wherein the FtsZ inhibitor and β-lactam antibiotic are administered within 1 hour of each other.

13. The method of claim 1, wherein the combination of said inhibitor of FtsZ and β-lactam antibiotic have a synergistic effect on antibacterial activity.

14. The method of claim 1, wherein the combination of said inhibitor of FtsZ and β-lactam antibiotic is effective to restore susceptibility of the methicillin-resistant Staphylococci to the β-lactam antibiotic.

15. A pharmaceutical composition comprising an effective amount of an inhibitor of FtsZ and a pharmaceutically acceptable carrier.

16. The pharmaceutical composition of claim 15 wherein the inhibitor of FtsZ inhibitor is selected from the group consisting of 2-carbamoyl pteridine, 534F6, A-189, amikacin, GTP, PC58538, PC170942, PC175515, PC175568, PC190723, SRI-3072, a zantrin, and analogs and pharmaceutically acceptable salts thereof.

17. The pharmaceutical composition of claim 15 wherein the inhibitor of FtsZ is selected from the group consisting of berberine, cinnamaldehyde, curcumin, dichamanetin, sanguinarine, taxane, totaro, viriditoxin, xanthoradone, and analogs and pharmaceutically acceptable salts thereof.

18. The pharmaceutical composition of claim 15 further comprising an effective amount of a β-lactam antibiotic.

19. The pharmaceutical composition of claim 18, wherein the β-lactam antibiotic is a carbapenem, cephalosporin, monolactam or penicillin.

20. The pharmaceutical composition of claim 18, wherein the β-lactam antibiotic is a carbapenem antibiotic.

21. The pharmaceutical composition of claim 20, wherein the carbapenem antibiotic is imipenem or ertapenem.

22. The pharmaceutical composition of claim 15 which further comprises a β-lactamase inhibitor.

23. (canceled)

24. (canceled)

25. (canceled)
